SEQUENCE LISTING (13 sequences)

SEQ ID NO: 1 - RNA, 19 nt, Mus musculus
  source 1..19: mol_type = genomic RNA
ggcacaaattttatgcctg

SEQ ID NO: 2 - RNA, 19 nt, Mus musculus
  source 1..19: mol_type = genomic RNA
caggcataaaatttgtgcc

SEQ ID NO: 3 - RNA, 19 nt, Homo sapiens
  source 1..19: mol_type = genomic RNA
cccatttagcacagaagtt

SEQ ID NO: 4 - RNA, 19 nt, Homo sapiens
  source 1..19: mol_type = genomic RNA
aacttctgtgctaaatggg

SEQ ID NO: 5 - RNA, 19 nt, synthetic construct
  source 1..19: mol_type = other RNA
caatattgcagctattcgg

SEQ ID NO: 6 - RNA, 19 nt, synthetic construct
  source 1..19: mol_type = other RNA
ccgaatagctgcaatattg

SEQ ID NO: 7 - DNA, 20 bp, synthetic construct
  source 1..20: mol_type = other DNA
gcactgctggattaccatcg

SEQ ID NO: 8 - DNA, 22 bp, synthetic construct
  source 1..22: mol_type = other DNA
gccacatagtaggggaagacag

SEQ ID NO: 9 - DNA, 19 bp, synthetic construct
  source 1..19: mol_type = other DNA
cactgctcgactaccaccg

SEQ ID NO: 10 - DNA, 22 bp, synthetic construct
  source 1..22: mol_type = other DNA
ccacaggaagtagatcacggag

SEQ ID NO: 11 - DNA, 32 bp, synthetic construct
  source 1..32: mol_type = other DNA
catgatcagctgggccaagaggcacaaatttt

SEQ ID NO: 12 - DNA, 16 bp, synthetic construct
  source 1..16: mol_type = other DNA
  modified_base 1: mod_base = OTHER; note = Locked nucleic acid
  modified_base 4: mod_base = OTHER; note = Locked nucleic acid
caggcataaaatttgt

SEQ ID NO: 13 - DNA, 20 bp, synthetic construct
  source 1..20: mol_type = other DNA
catgatcagctgggccaaga

(19) United States
(12) Patent Application Publication
(10) Pub. No.: US 2025/0084416 A1
(43) Pub. Date: Mar. 13, 2025

(54) TARGETING OF XKR8 IN THERAPIES

(71) Applicant: University of Pittsburgh - Of the Commonwealth System of Higher Education, Pittsburgh, PA (US)

(72) Inventors: Song Li, Mars, PA (US); Yuang Chen, Cambridge, MA (US)

(21) Appl. No.: 18/728,870

(22) PCT Filed: Sep. 19, 2022
(86) PCT No.: PCT/US2022/043940

§ 371 Date: Jul. 13, 2024

Related U.S. Application Data
(60) Provisional application No. 63/299,466, filed on Jan. 14, 2022.

(51) Int. Cl. C12N 15/113; A61K 47/60; A61K 47/61; A61K 47/64

(52) U.S. Cl. CPC C12N 15/1137; A61K 47/60; A61K 47/61; A61K 47/64; A61K 47/6435; C12N 2310/14; C12N 2310/351; C12N 2310/3513

(57) ABSTRACT

A therapeutic system or combination includes a first therapeutic agent to treat a disease condition and a second therapeutic agent adnrinistrable within a predetermined time of administration of the first therapeutic agent the second therapeutic agent inhibiting the function of Xkr8.

### CROSS-REFERENCE TO RELATED APPLICATIONS

This application claims benefit of U.S. Provisional Patent Application Ser. No. 63/299,466, filed, filed Jan. 14, 2022, the disclosure of which is incorporated herein by reference.

### GOVERNMENTAL INTEREST

This invention was made with government support under grant number CA223788 awarded by the National Institutes of Health. The government has certain rights in the invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Phosphatidylserine (PS) has been reported to be implicated in cancer immunosuppression. As an important component of membrane structure, PS is confined to the inner leaflet. This asymmetric distribution is maintained by the action of P4-ATPases such as ATP11A and 11C, which function as flippases in the plasma membrane to actively translocate PS from the outer leaflet to the inner leaflet. Scramblases collapse membrane asymmetry, thereby randomizing all phospholipid species between leaflets, which effectively increases the accumulation of PS on the external side of the membrane.

The scramblase Xkr8 carries a caspase 3 recognition site in its C-terminal region. It is generally believed that Xkr8 is regulated post-transcriptionally and is activated by caspases during apoptosis.

Flippases are inactivated by caspases, resulting in irreversible exposure of PS on the cell surface. PS exposure is not limited to apoptosis. It is also seen in coagulation, myoblast fusion, and T lymphocyte activation.

Macrophages engulf apoptotic cells but not living cells, and PS serves as an important “eat me” signal. The engagement of PS on apoptotic cells with its receptors on immune cells leads to profound immunosuppression. Moreover, this evolutionally conserved mechanism is important for the silent clearance of apoptotic cells in normal animal development. PS also functions as an upstream immune checkpoint that contributes to the immunosuppressed tumor microenvironment by preventing immune reactions.

The discovery of PS receptors and the underlying signaling suggests a strategy for cancer immunotherapy through blocking the immunosuppressive pathway. However, such strategy may also negatively affect the antitumor immunity of T cells. Blockade of PS via annexin AS (AnxA5) or PS-specific antibody (Ab) is another attractive strategy. A family of PS-targeting antibodies has been developed. Unfortunately, despite the demonstrated efficacy and safety in preclinical and phase I/II clinical studies, a larger phase III trial failed to demonstrate benefit over chemotherapy alone.

Another concern is the targeting of the antibody to other PS-expressing cells under physiological condition and/or resulting from comorbid diseases such as cardiovascular diseases, especially in combination with systemic chemotherapy. Strategies that specifically target PS on tumor cells but do not affect PS on normal cells, and those that prevent or decrease the quantity of PS exposure when combined with other therapeutic/anticancer drugs, may represent a more attractive approach.

### SUMMARY

[In one aspect, a therapeutic system or combination includes a first therapeutic agent to treat a disease condition and a second therapeutic agent to be administered within a predetermined time of administration of the first therapeutic agent, the second therapeutic agent inhibiting the function of Xkr8. The first therapeutic agent may trigger a therapeutic response which induces Xkr8 expression in addition to activation of Xkr8. In a number of embodiments, the second therapeutic agent functions via RNA interference. The second therapeutic agent may, for example, include (for example, in a conjugate) or be Xkr8 siRNA. In a number of embodiments, the first therapeutic agent is a chemotherapeutic agent.

The therapeutic system may include nanostructures formed from self-assembly of a plurality of amphiphilic polymers including cationic groups, wherein a plurality of the first therapeutic agent, which is hydrophobic or lipophilic (for example, a hydrophilic or lipophilic chemotherapeutic agent), is associated with a core of the nanostructure and the second therapeutic agent including Xkr8 siRNA is added to the nanostructures and is associated with the cationic groups thereof. The nanostructures may further include a coating, application, or layer added to the nanostructures, which includes a negatively charged targeting agent (that is, an agent which actively targets a region of interest such as a tumor). The coating, application, or layer need not be continuous. The negative charge provides anchoring interaction via charge-charge interactions with cationic groups of the amphiphilic polymers forming the nanostructures. In addition to providing targeting, such negatively charged agents assist in charge neutralization/shielding of positive charge to achieve a nanostructure/nanocarrier exhibiting approximate charge neutrality. The coating, application, or layer may further include a hydrophilic polymeric compound which may, for example, include a negative charge to anchor the hydrophilic polymeric compound to the cationic groups via charge-charge interaction. The hydrophilic polymeric compound may provide further charge neutralization and may, in some embodiments, provide a degree of shielding for the targeting agent.

In a number of embodiments, the negatively charged targeting agent includes or is a ligand for a cell receptor, a peptide, an aptamer, a polysaccharide, or an antibody. The negative charge may be inherent in the targeting agent or be added thereto (for example, via conjugation with a negatively charged molecule or compound). In a number of representative embodiments hereof, the targeting agent is a negatively charged CD44 ligand. In a number of embodiments, the application, which is added to the nanostructures, comprises a negative-charged receptor ligand such as a negatively charged CD44 ligand and a hydrophilic polymeric compound. The hydrophilic polymeric compound may include a negative charge as described above. In a number of embodiments, the hydrophilic polymeric compound includes or is a conjugate of a negatively charged molecule or compound and a hydrophilic polymer. The negatively charged molecule or compound which is conjugated to the hydrophilic polymer may be the targeting agent (for example, a CD44 ligand).

CD44 ligand(s) hereof may, for example, include osteopontin, a collagen, a matrix metalloproteinase, chondroitin sulfate, hyaluronic acid, or a derivative thereof. In a number of embodiments, the CD44 ligand is chondroitin sulfate.

In a number of embodiments, the second therapeutic agent including Xkr8 siRNA is added to the nanostructures before application of the negatively charged targeting agent (for example, a negatively charged CD44 ligand) and (in embodiments wherein the application further includes the hydrophilic polymeric compound) the hydrophilic polymeric compound. The targeting agent and the hydrophilic polymeric compound may be added as a mixture.

The first therapeutic agent may be a small molecule therapeutic compound. The first therapeutic agent may, for example, have a molecular weight below 1 kDa.

As used herein, the “cationic group” refers to an inherently cationic group or a group which forms a cation in vivo. In a number of embodiments, the group which forms a cation in vivo is an amine group, wherein the amine group is an acyclic amine group, a cyclic amine group or a heterocyclic amine group. In a number of embodiments, the amine group is selected from the group consisting of a metformin group, a morpholine group, a piperazine group, a pyridine group, a pyrrolidine group, piperidine, a thiomorpholine, a thiomorpholine oxide, a thiomorpholine dioxide, an imidazole, a guanidine, a biguanidine or a creatine.

The hydrophilic polymer may, for example, be selected from the group consisting of a polyalkylene oxide, a polyvinylalcohol, a polyacrylic acid, a polyacrylamide, a polyoxazoline, a polysaccharide and a polypeptide. In a number of embodiments, the hydrophilic polymer is polyethylene glycol.

A ratio of the negatively charged targeting agent to the hydrophilic polymeric compound added to the nanostructures may be determined such that uptake of the nanostructures at one or more regions other than the region of interest is maintained at a sufficiently low level to allow interaction of the negatively charged targeting agent at the region of interest (for example, a tumor). In a number of embodiments, a ratio of the negatively charged CD44 ligand to the hydrophilic polymeric compound is determined such that uptake of the nanostructures in the liver of a patient is maintained at a sufficiently low level to allow interaction of the negatively charged CD44 ligand with CD44 on a tumor remote from the liver.

In a number of embodiments, each of the plurality of amphiphilic polymers comprises a hydrophobic polymer backbone, a first plurality of pendant groups attached to the hydrophobic polymer backbone and comprising at least one of the cationic groups, and a second plurality of pendant groups attached to the hydrophobic polymer backbone and comprising at least one hydrophilic polymer. The hydrophobic polymer backbone may further include a pendant lipidic group. The hydrophobic polymer backbone may be formed via a free radical polymerization. The hydrophobic polymer backbone may be formed via a reversible-deactivation radical polymerization.

In another aspect, a method of delivering a combination therapy to treat a disease condition includes administering a first therapeutic agent to treat the disease condition and administering a second therapeutic agent within a predetermined time of administering the first therapeutic agent, the second therapeutic agent inhibiting the expression or function of Xkr8. The first therapeutic agent may induce a therapeutic response which induces the expression of Xkr8 in addition to activation Xkr8. In a number of embodiments, the second therapeutic agent functions via RNA interference. The second therapeutic agent may include or be Xkr8 siRNA. In a number of embodiments, the first therapeutic agent is a chemotherapeutic agent.

The combination therapy may be delivered via nanostructures formed from self-assembly of a plurality of amphiphilic polymers comprising cationic groups, wherein a plurality of the first therapeutic agent, which is hydrophobic or lipophilic, is associated with a core of the nanostructure and the second therapeutic agent including Xkr8 siRNA is added to the nanostructures and is associated with the cationic groups thereof. The nanostructures may further include a coating, application, or layer added to the nanostructures, the application including a negatively charged targeting compound (for example, a CD44 ligand) as described above. The application may further include a hydrophilic polymeric compound. The hydrophilic polymeric compound may include a negative charge. In a number of embodiments, the hydrophilic polymeric compound includes a conjugate of a negatively charged molecule or compound and a hydrophilic polymer. In a number of embodiments, the negatively charged targeting agent is a CD44 ligand and the negatively charged molecule or compound which is conjugated to the hydrophilic polymer may be a CD44 ligand. CD44 ligands hereof may, for example, be osteopontin, a collagen, a matrix metalloproteinase, chondroitin sulfate, hyaluronic acid, or a derivative thereof.

In a further aspect, a formulation includes nanostructures formed from self-assembly of a plurality of amphiphilic polymers, a plurality of a first hydrophobic or lipophilic therapeutic agent associated with a core of each of the nanostructures, and a second therapeutic agent which inhibits the expression or function of Xkr8. The plurality of amphiphilic polymers may, for example, include cationic groups. The second therapeutic agent may, for example, include or be Xkr8 siRNA which is added to the nanostructures to be associated with the cationic groups thereof. The nanostructures may further include a coating, application, or layer added to the nanostructures, wherein the application includes a negatively charged targeting agent as described above. The application may further include a hydrophilic polymeric compound. As described above, the hydrophilic polymeric compound may include a negative charge. In a number of embodiments, the hydrophilic polymeric compound includes a conjugate of a negatively charged molecule or compound and a hydrophilic polymer. The negatively charged molecule or compound, which is conjugated to the hydrophilic polymer may, for example, be a targeting agent (for example, a CD44 ligand). As further described above, the CD44 ligands hereof may, for example, include osteopontin, a collagen, a matrix metalloproteinase, chondroitin sulfate, hyaluronic acid, or a derivative thereof. In a number of embodiments, the CD44 ligand is chondroitin sulfate. The second therapeutic agent including Xkr8 siRNA may, for example, be added to the nanostructures before application of the negatively charged targeting agent such as a CD44 ligand and (in embodiment wherein the application further include a hydrophilic polymeric compound) before a combination of the negatively charged targeting agent and a hydrophilic polymeric compound.

The first therapeutic agent may, for example, be a small molecule therapeutic compound. In a number of embodiments, the first therapeutic agent has a molecular weight below 1 kDa.

As described above, the cationic groups may be inherently cationic groups or groups which form cations in vivo. In a number of embodiments, the group which forms a cation in vito is an amine group, wherein the amine group is an acyclic amine group, a cyclic amine group or a heterocyclic amine group. In a number of embodiments, the amine group is selected from the group consisting of a metformin group, a morpholine group, a piperazine group, a pyridine group, a pyrrolidine group, piperidine, a thiomorpholine, a thiomorpholine oxide, a thiomorpholine dioxide, an imidazole, a guanidine, a biguanidine or a creatine.

In a number of embodiments, the hydrophilic polymer is selected from the group consisting of a polyalkylene oxide, a polyvinylalcohol, a polyacrylic acid, a polyacrylamide, a polyoxazoline, a polysaccharide and a polypeptide. In a number of embodiments, the hydrophilic polymer is polyethylene glycol.

A ratio of the negatively charged targeting agent to the hydrophilic polymeric compound added to the nanostructures may be determined such that uptake of the nanostructures at one or more regions other than the region of interest is maintained at a sufficiently low level to allow interaction of the negatively charged targeting agent at the region of interest (for example, a tumor). In a number of embodiments, a ratio of the negatively charged CD44 ligand to the hydrophilic polymeric compound may be determined such that uptake of the nanostructures in the liver of a patient is maintained at a sufficiently low level to allow interaction of the negatively charged CD44 ligand with CD44 on a tumor remote from the liver.

In a number of embodiments, each of the plurality of amphiphilic polymers includes a hydrophobic polymer backbone, a first plurality of pendant groups attached to the hydrophobic polymer backbone and including at least one of the cationic groups, and a second plurality of pendant groups attached to the hydrophobic polymer backbone and comprising at least one hydrophilic polymer. The hydrophobic polymer backbone may further include a pendant lipidic group. In a number of embodiments, the hydrophobic polymer backbone is formed via a free radical polymerization. In a number of embodiments, the hydrophobic polymer backbone is formed via a controlled/living radical polymerization or a reversible-deactivation radical polymerization.

In still a further aspect, a method of providing treatment to a patient includes administering a first therapeutic agent to treat a disease condition and administering a second therapeutic agent delivered within a predetermined time of administering the first therapeutic agent, wherein the second therapeutic agent inhibiting the expression or function of Xkr8. The first therapeutic agent may, for example, induce a therapeutic response which induces the expression of Xkr8 in addition to activation of Xkr8. In a number of embodiments, the second therapeutic agent functions via RNA interference. The second therapeutic agent may, for example, include or be Xkr8 siRNA. In a number of embodiments, the first therapeutic agent is a chemotherapeutic agent.

In a number of embodiments, the first therapeutic agent and the second therapeutic agent are administered via nanostructures formed from self-assembly of a plurality of amphiphilic polymers comprising cationic groups, wherein a plurality of the first therapeutic agent, which is hydrophobic or lipophilic, is associated with a core of the nanostructure and the Xkr8 siRNA is added to the nanostructures and is associated with the cationic groups thereof. The first therapeutic agent and the second therapeutic agent may, for example, be delivered in a formulation as described above and elsewhere herein.

The present devices, systems, methods and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1a illustrates a volcano plot for the RNA-seq analysis of CT26 tumors in mice treated with FuOXP NPs versus empty NPs.

FIG. 1b illustrates qRT-PCR analysis of mXkr8 mRNA expression in CT26 tumors in mice treated with empty NPs or FuOXP NPs. N=5, ns: not significant, P>0.05, ***P<0.001.

FIG. 1c: illustrates qRT-PCR analysis of Xkr8 mRNA expression in various types of cultured tumor cells at 24 h following treatment with FuOXP, DOX, and PTX, respectively. N=3, ***P<0.001.

FIG. 1d illustrates Western analysis of hXkr8 protein expression in PANC-1 or HT29 tumor cells at 24 h following treatment with FuOXP, DOX, and PTX, respectively. hXkr8 MW: 45 kDa, β-Tubulin MW: 55 kDa.

FIG. 1e illustrates densitometry analysis of protein bands in FIG. 1d. N=3, ***P<0.001.

FIGS. 1f and 1g illustrate qRT-PCR analysis of Xkr8 mRNA expression levels over time in CT26 (1f) and PANC-1 (1g) tumor cells following 12 h of FuOXP treatment and then actinomycin D treatment for 2 or 4 h. N=3, ns: not significant.

FIGS. 1h-k illustrate changes in the expression levels of Xkr8 mRNA in CT-26 (1h) and PANC-1(1i), and Xkr8 protein (human) in PANC-1 cells (FIGS. 1j & 1k) treated with N-acetylcysteine (NAC) for 12 h followed by treatment with different drugs for another 12 h, and wherein FIG. 1k illustrates densitometry analysis of protein bands in Western blot (FIG. 1j). N=3, ***P<0.00.1.

FIG. 2a illustrates a schematic diagram of an embodiment of a protocol for the preparation of FuOXP/siRNA-coloaded PMBOP-CP NPs.

FIG. 2b illustrates CMC of PMBOP polymer.

FIG. 2c illustrates biophysical characterization of PMBOP/FuOXP mixed micelles at various carrier/drug ratios (w/w). N=3, results are presented as the mean SEM.

FIG. 2d illustrates gel retardation assay of PMBOP/FuOXP/siRNA complexes at various N/P ratios.

FIG. 2e illustrates sizes and zeta potentials of PMBOP/FuOXP/siRNA complexes at various N/P ratios.

FIG. 2f illustrates sizes and zeta potentials of FuOXP/siRNA-coloaded PMBOP-C NPs (coated with CS alone) at various N/P/S ratios.

FIG. 2g illustrates sizes and zeta potentials of FuOXP/siRNA-coloaded PMBOP-CP NPs (coated with a mixture of CS and PEG-CS) at various N/P/S(CS)/S(PEG-CS) ratios. N=3 (FIGS. 1e-1f).

FIG. 2h illustrates spherical morphology of PMBOP-CP NPs with and without coloaded siRNA by cryo-EM. Scale bar, 100 nm.

FIG. 2i illustrates cumulative FuOXP release profile from PMBOP-CP NPs with or without siRNA complexation in PBS or murine serum. N=3, *P<0.05, **P<0.01.

FIG. 2j illustrates protection of siRNA in PMBOP-CP NPs against the digestion by RNAse.

FIGS. 3a-3b illustrates fluorescence microscopic examination of tumor and liver sections at 24 h following i.v. injection of Cy5.5-siXkr8-loaded PMBOP NPs of different N/P/S(CS)/S(PEG-CS) ratios.

FIG. 3c illustrates NIR whole body imaging of CT26 tumor-bearing mice at 24 h following i.v. administration of Cy5.5-siXkr8-loaded PMBOP-CP NPs.

FIG. 3d illustrates ex vivo imaging of tumors (CT26) and major organs at 24 h following i.v. administration of Cy5-siXkr8-loaded PMBOP-CP NPs.

FIG. 3e illustrates fluorescence (Cy5.5-siXkr8) intensity at tumors and liver at different times following i.v. administration of Cy5.5-siXkr8-loaded PMBOP-CP NPs. N=3, **P<0.01, ***P<0.001.

FIGS. 3f and 3g illustrate changes in fluorescence (Cy5.5-siXkr8) intensity in blood at different times following iv. administration of free Cy5.5-siXkr8 or Cy5.5-siXkr8-loaded PMBOP-CP NPs. N=3, ***P<0.001.

FIGS. 3h and 3i illustrate ex vvo imaging of tumors and major organs at 24 h following i.v. administration of Cy5.5-siXkr8-loaded PMBOP-CP NPs in various types of s.c. tumor models (FIG. 3h), and a CT26 orthotopic model (FIG. 3i).

FIG. 3j illustrates a CT-26 lung metastasis model.

FIGS. 3k-3n illustrate confocal laser scanning microscopic images of tumor (s.c. CT26) sections at 24 h following i.v. administration of Cy5.5 siXkr8-loaded PMBOP-CP NPs, wherein: FIG. 3k illustrates a merged image of cell nuclei (Hoechst), F-actin (Alexa Fluor® 488 Phalloidin) and siRNA (Cy5.5) at a 20× magnification. Scale bar, 30 μm, FIG. 3l illustrates the 3D Z-stacking of two layers of 2D scanning images from different depths. Green: 0.1 μm layer from the first scan (FIG. 3m). Red: 0.5 μm layer from the first scan (FIG. 3m). Magnification, 600×. Scale bar, 1 μm. large white arrow: punctuated pattern. Small white arrow: diffused pattern.

FIGS. 4A and 4b illustrate NIR whole body (FIG. 4a) and ex vivo (FIG. 4b) imaging of CT26 tumor targeting in WT and CD44^{-/-} mice at 24 h following i.v. administration of Cy5.5-siXkr8-loaded PMBOP-CP NPs.

FIG. 4c illustrates ex vivo imaging of blood collected from WT and CD44^{-/-} mice bearing CT26 tumors.

FIGS. 4d and 4e illustrate fluorescence (Cy5.5-siXkr8) intensity in tumors and liver (FIG. 4d), and in blood (FIG. 4e) at 24 h following i.v. administration of the NPs. N=3, ***P<0.001.

FIG. 4f illustrates NIR whole body imaging of CT26 tumor-bearing Zombie mice at 24 h following i.v. administration of the NPs.

FIG. 4g illustrates fluorescence (Cy5.5-siXkr8) intensity in tumors and liver at 24 h following i.v. administration of the NPs. N=3, ***P<0.001.

FIG. 4h illustrates flow analysis of CD44 expression in mouse LSECs (WT and CD44^{-/-}), T_{q}, Tₐ, HUVEC_{q}, HLIVECₐ and CT26 cells. N=3, *P<0.05, ***P<0.001.

FIG. 4i illustrates quantitative analysis of the % of Cy5.5⁺ cells and the MFI at 4 h following incubation of Cy5.5-siXkr8-loaded PMBOP-CP NPs with LSECs (WT and CD44^{-/-}), HUVEC_{q}, and HUVECₐ cells, respectively. N=3, **P<0.01.

FIG. 4j illustrates flow analysis of Cy5.5⁺ CT26 cells (in lower chamber) at 12 h following addition of Cy5.5-siXkr8-loaded PMBOP-CP NPs to HUVECs (upper chamber) in a Transwell plate. N=3, ***P<0.001.

FIG. 4k illustrates quantitative analysis of cellular uptake of Cy5.5-siXkr8-loaded PMBOP-CP NPs by CT26, HUVECₐ, T_{q}, and Tₐ cells. N=3, ***P<0.001.

FIG. 4l illustrates an embodiment of a proposed model of PMBOP-CP NPs-mediated tumor targeting through both EPR and transcytosis. NPs coated with CS alone were rapidly eliminated by LSECs/CD44-mediated liver uptake with limited accumulation at tumors. Adding to the NPs an optimized amount of CS/PEG-CS led to significant decrease of uptake by LSECs without significantly affecting the interaction with tumor ECs, resulting in effective tumor targeting.

FIG. 5a illustrates ICP-MS analysis of plasma concentrations of Pt after tail vein injection of free FuOXP/siXkr8 or FuOXP/siXkr8 NPs in naïve BALB/c mice. The dose was 5 and 1 mg/kg for FuOXP and siXkr8, respectively. N=3, ***P<0.001.

FIG. 5b illustrates pharmacokinetic parameters of Pt that were analyzed by a non-compartmental model. N=3. Results are presented as the mean±SEM.

FIGS. 5c and 5d illustrates biodistribution of Pt in different organs in BALB/c mice bearing CT26 tumors at different times following i.v. injection of FuOXP/siXkr8 NPs. N=3. **P<0.01, ***P<0.001.

FIG. 5e illustrates qRT-PCR analysis of plasma concentrations of siXkr8 following i.v. injection of free FuOXP/siXkr8 or FuOXP/siXkr8 NPs in naïve BALB/c mice. N=3. ***P<0.001.

FIG. 5f illustrates pharmacokinetic parameters of siXkr8 were analyzed by a non-compartmental model. N=3. Results are presented as the mean±SEM.

FIGS. 5g and 5h illustrate biodistribution of siXkr8 in different organs in BALB/c mice bearing CT26 tumors at different times following i.v. injection of FuOXP/siXkr8 NPs. N=3. *P<0.05, ***P<0.001.

FIG. 5i illustrates fluorescence microscopic images of cultured CT26 tumors cells at 2 h following treatment with Cy5.5-siXkr8-loaded PMBOP-CP NPs.

FIG. 5j illustrates efficiency of siRNA PMBOP-CP NPs-mediated gene knockdown in cultured MC38-Luc cells. MC38-Luc cells were pretreated with various endocytosis inhibitors for 1 h followed by treatment with luciferase siRNA NPs. Twenty-four h later, luciferase activity in cells receiving various treatments was examined. N=3. Results are presented as the mean±SEM.

FIG. 5k illustrates qRT-PCR analysis of Xkr8 mRNA expression levels in CT26 cells 24 h following treatment with siCT NPs, siXkr8 Lipo (Lipofectamine RNAiMAX transfection reagent) or siXkr8 NPs. N=3, *P<0.05. ***P<0.001.

FIG. 5l illustrates C57BL/6 mice bearing MC38-Luc tumors that received i.v. administration of luciferase siRNA NPs at a dose of 2 mg/kg once every 5 days and the mice were subjected to whole body bioluminescence imaging the next day after each treatment. N=3, ns: not significant, ***P<0.001.

FIG. 5m illustrates CT26 tumor-bearing mice that were treated with siXkr8 NPs or FuOXP/siXkr8-coloaded NPs at a FuOXP dose of 5 mg/kg and siRNA dose of 1 mg/kg once every 5 days. The mRNA expression levels of Xkr8 in tumors were examined by qRT-PCR the next day after the last treatment. N=5, *P<0.05, ***P<0.001.

FIG. 6a illustrates studies of CT26 tumor cells that were treated with control or siXkr8 NPs for 72 h followed by treatment with FuOXP/siRNA-coloaded NPs. The numbers of Annexin V⁺ cells were examined by flow 24 h later. N=3, ***P<0.001.

FIG. 6b illustrates studies of CT26 tumor cells which received similar treatments as described in FIG. 6a and the amount of secreted Annexin V⁺ EVs was examined by flow. N=3, **P<0.01, ***P<0.001.

FIG. 6c illustrates studies of CT26 tumor cells which received similar treatment as described in FIG. 6a and the tumor cells were then co-cultured with primary macrophages. The percentages of M1 and M2 cells were then quantified based on the flow analysis of various M1/M2 surface markers. N=3, **P<0.01.

FIGS. 6d and 6e illustrates studies of mice bearing CT26 (FIG. 6d) or Panc02 (FIG. 6e) tumors which received various treatments once every 5 days for 3 times at a siRNA dose of 1 mg/kg and FuOXP dose of 5 mg/kg. Tumor volumes were followed once every 2 days. N=5, **P<0.01, ***P<0.001.

FIGS. 6f-6k illustrate studies of CT26 tumor-bearing mice which received various treatments as described in FIG. d. Single cell suspensions were prepared at the completion of therapy study and subjected to various flow analysis including Annexin V⁺ cells (FIG. 6f), CD45⁺ cells (FIG. 6g), Treg cells (FIG. 6h), M1/M2-like ratios (i), IFNγ⁺ CD8+ cells (FIG. 6j), and GzmB CD8⁺ cells (FIG. 6k), respectively. N=5, ns: not significant, P>0.05, *P<0.05, **P<0.01, ***P<0.001.

FIG. 6l illustrates studies of Panc02 tumor-bearing mice which received various treatments as described in FIG. e. Single cell suspensions were prepared at the completion of therapy study and subjected to flow analysis of PD-1⁺ CD8⁺ cells. N=5, **P<0.01.

FIGS. 6m and 6n illustrate studies of mice bearing Panc02 tumors which received various treatments when the tumors reached ˜155 mm³ in sizes once every 5 days for 3 times at a dose of 1, 5 and 10 mg/kg for siXkr8, FuOXP, and anti-PD-1, respectively. Tumor growth (FIG. 6m) and survival (FIG. 6n) were followed. N=8, **P<0.01, ***P<0.001.

FIG. 7a illustrates studies of CT26 tumor-bearing mice which received the treatments as described in FIG. 6d. Mouse weights were measured once every 2 days.

FIG. 7b illustrates serum levels of AST and ALT at the completion of the therapy study. N=3, ns: not significant, P>0.05.

FIG. 7c illustrates histology of major organs in mice receiving different treatments as described in FIG. 6d.

FIG. 7d illustrates serum levels of TNF-α and IL-6 at 2 h following i.v. administration of siRNA PMBOP-CP NPs or siRNA complexed with DOTAP liposomes (N/P, 10/1) at a siRNA dose of 1 mg/kg. N=3, ns: not significant, P>0.05, ***P<0.001.

FIGS. 7e and 7f illustrate that FuOXP NPs caused minimal changes in Xkr8 mRNA levels (FIG. 7e) and PS+ cells (FIG. 7f) in liver.

FIG. 7g illustrates that minimal changes in CD45+ cells in liver were caused by FuOXP/siXkr8 NPs. N=3, ns: not significant.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a therapeutic agent” includes a plurality of such therapeutic agent and equivalents thereof known to those skilled in the art, and so forth, and reference to “the therapeutic agent” is a reference to one or more such therapeutic agents and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

In a number of embodiments hereof, a first therapeutic agent is administered or delivered to treat a disease condition, and a second therapeutic agent is administered or delivered within a predetermined time (including simultaneously, contemporaneously or at different times) of the first therapeutic agent. The second therapeutic agent inhibits the expression or function of Xkr8. The second therapeutic agent may, for example, inhibit the expression or function of Xkr8 at the genome level, at the mRNA level, or at the protein level. Gene silencing therapies may, for example, be used during either transcription or translation via, for example, small-molecule therapy, nucleotide-based therapies, CRISPR, etc. In post-transcriptional silencing, RNA interference (RNAi) may, for example, be effected or achieved using small molecules, microRNA (miRNA) and/or small interfering RNA (siRNA). Antisense oligonucleotides may be used to target mRNA. Likewise, ribozymes may be used to target mRNA. Protein-level therapies may, for example, be effected or achieved using small molecules and targeted protein degradation. The first therapeutic agent may, for example, induce a therapeutic response which induces Xkr8 expression in addition to activation of Xkr8. For example, studies hereof have led to the discovery that treatment of tumors with chemotherapeutic drugs led to significant upregulation of Xkr8 mRNA in vitro and in vivo, indicating that Xkr8 may serve as a novel therapeutic target for cancer treatment. In the case of a combination treatment in which a first therapeutic agent is delivered to treat a disease condition and a second therapeutic agent is delivered to inhibit the expression or function of Xkr8, the second therapeutic agent may be delivered within a time period determined to provide inhibition of the upregulation/activation resulting from the therapeutic effect of the first therapeutic agent. In a number of embodiments, the first therapeutic agent and the second therapeutic agent are delivered via a common carrier which may be a nanostructure-based carrier or nanocarrier. Various nanocarriers based upon self-assembling amphiphilic polymers are, for example, described in U.S. Pat. Nos. 10,172,795 and 9,855,341, U.S. Patent Publication Nos. 2018/0214563 and 2021/0236645, and in PCT International Patent Application No. PCT/US22/43938, filed Sep. 19, 2022, the disclosures of which are incorporated herein by reference. Depending upon the nature of the first therapeutic agent and the second therapeutic agent, one skilled in the art can choose or design a suitable carrier.

In a number of embodiments hereof, a novel nanocarrier was developed that is capable of codelivery of siRNA (and/or other polynucleotide-based (for example, a nucleic acid such as DNA, RNA, etc.) therapeutics) and small molecule drugs. This nanocarrier was extensively characterized with respect to the efficiency of tumor targeting and the underlying mechanism. Further, the therapeutic efficacy as well as the underlying mechanism of codelivery of siXkr8 and the representative anticancer agent 5-Fu/oxaliplatin was studied in syngeneic mouse models of colon and pancreatic cancers.

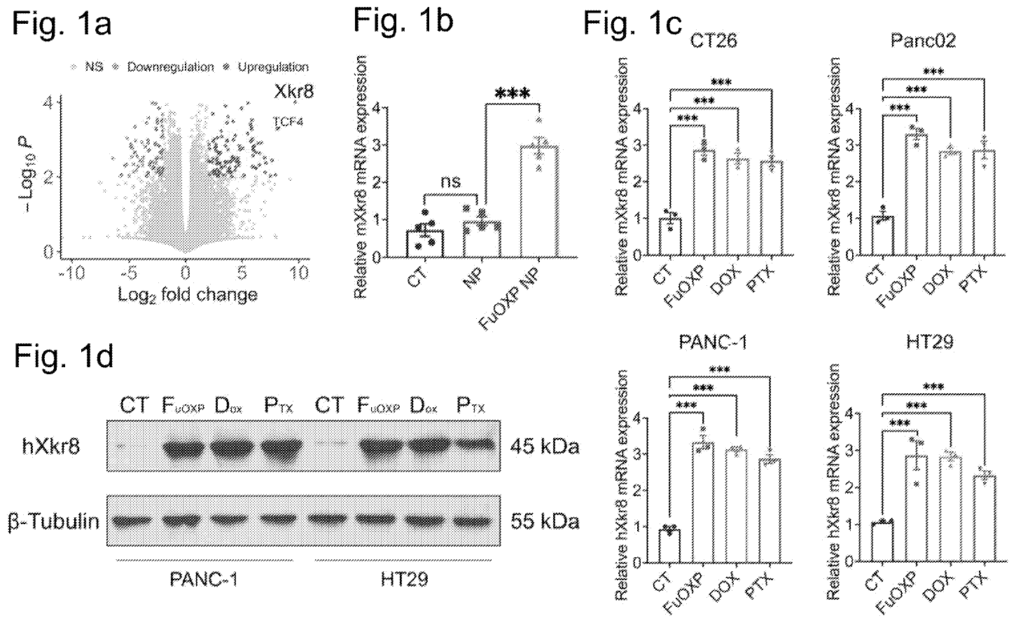

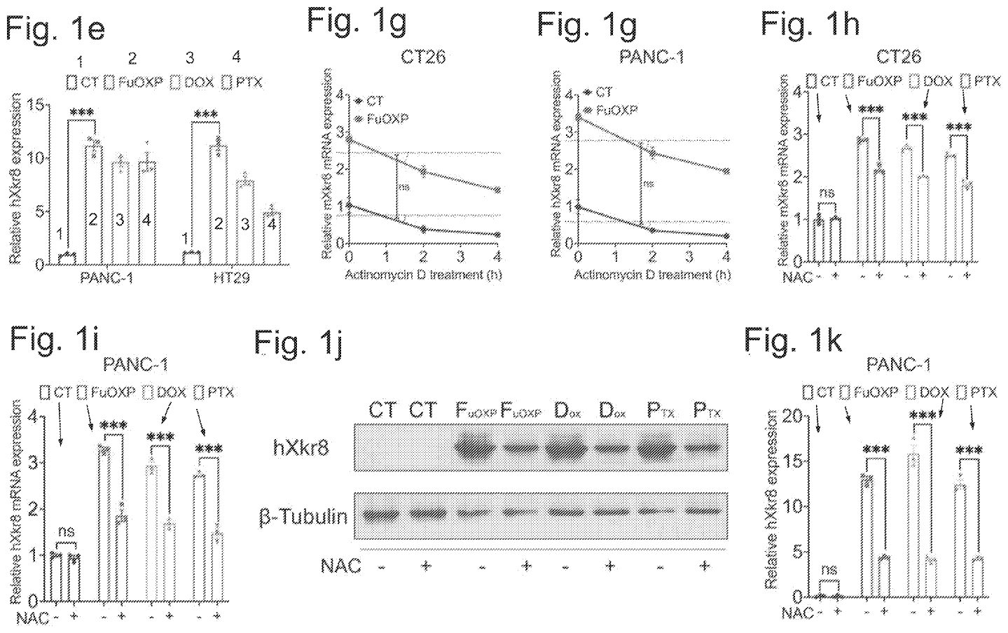

In independent studies to define the mechanism of chemoresistance in colorectal cancer (CRC), RNA-seq was conducted to examine changes in gene expression profile after treatment of CT26 tumor-bearing mice with FuOXP. FuOXP is a prodrug conjugate of 5-FU and oxoplatin previously reported to have improved antitumor activity and decreased cytotoxicity towards normal cells. CT26 is a syngeneic CRC model that responded poorly to moderately to 5-FU/OXP as well as FuOXP. As shown in FIG. 1a, Xkr8 was one of the top genes for which mRNA expression was most significantly upregulated. Induction of murine Xkr8 (mXkr8) mRNA expression in vivo was further confirmed by qRT-PCR (FIG. 1b). Induction of Xkr8 mRNA expression by FuOXP as well as two other drugs (doxorubicin—DOX, and paclitaxel—PTX) was also demonstrated in cultured murine and human CRC (CT26 and HT-29) and pancreatic cancer (PCa) (Panc02 and PANC-1) cell lines, respectively (FIG. 1c). Induction of the human Xkr8 (hXkr8) was confirmed at the protein level in human CRC (HT-29) and PCa (PANC-1) cell lines following treatment with the three drugs, respectively (FIGS. 1d and 1e). Induction of mXkr8 at the protein level could not be evaluated in murine cancer cells in vitro and in vivo as a result a lack of available antibodies.

FIG. 1f illustrates the kinetics of mXkr8 mRNA levels in CT26 cells following 12 h of FuOXP treatment followed by another 2 h of treatment with actinomycin D (ActD), a transcription inhibitor. Cells treated with ActD alone showed a gradual decrease in the level of mXkr8 mRNA, reflecting the normal rate of Xkr8 mRNA degradation in CT26 cells. Cells treated with FuOXP for 12 h showed a higher initial level of mXkr8 mRNA, consistent with the data shown in FIG. 1e. Nonetheless, cells receiving co-treatment showed similar kinetics of decline in the level of mXkr8 mRNA, indicating that FuOXP did not affect the rate of mXkr8 mRNA degradation. Similar results were observed in PANC-1 cells (FIG. 1g). The data indicated that FuOXP, and potentially other drugs as well, caused Xkr8 induction through enhanced gene transcription.

The mechanism of Xkr8 induction by different chemotherapeutic drugs is not fully determined. Preliminary studies indicated that pretreatment with the antioxidant N-acetylcysteine (NAC) partially attenuated the Xkr8 induction by the 3 drugs in CT26 (FIG. 1h) and PANC-1 cells (FIG. 1i-k) at both mRNA (FIG. 1h-i) and protein (FIG. 1j-k) levels. Without limitation to any mechanism, such results suggest a role of oxidative stress in Xkr8 induction. This observation is consistent with a previous report that oxidative stress might be involved in the exposure of PS in tumor vasculature.

In a number of studies hereof, the expression level of Xkr8 is downregulated through siRNA-mediated knockdown. Additionally, a representative nanocarrier. PMBOP-CP, was developed to achieve codelivery of SiXkr8 and FuOXP. Nanocarriers suitable for use herein, including PMBOP-CP, are described in PCT International Patent Application No. PCT/US22/43938, which is assigned to the assignee hereof.

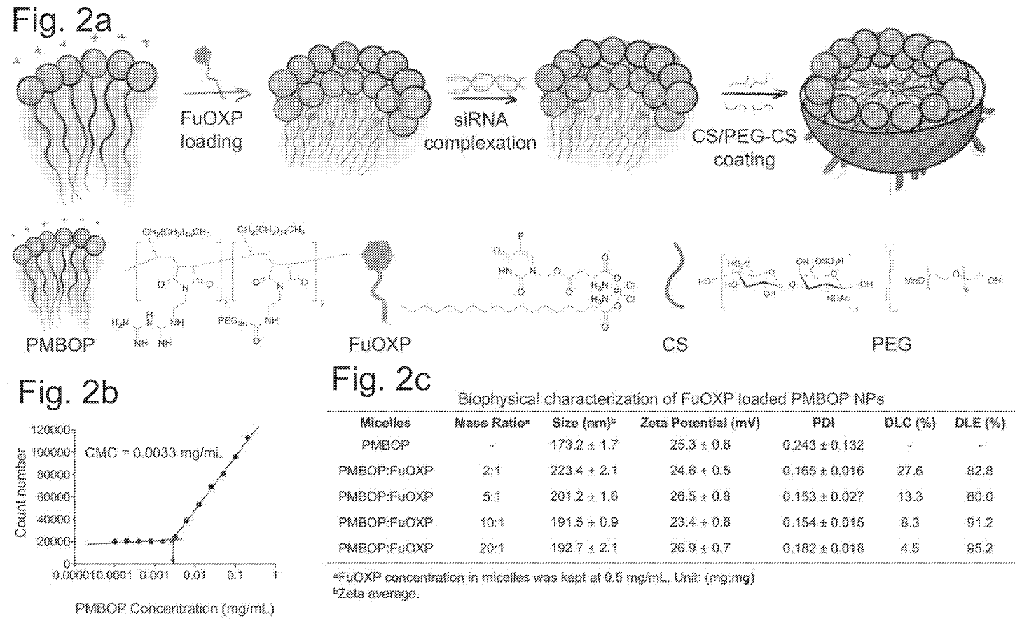

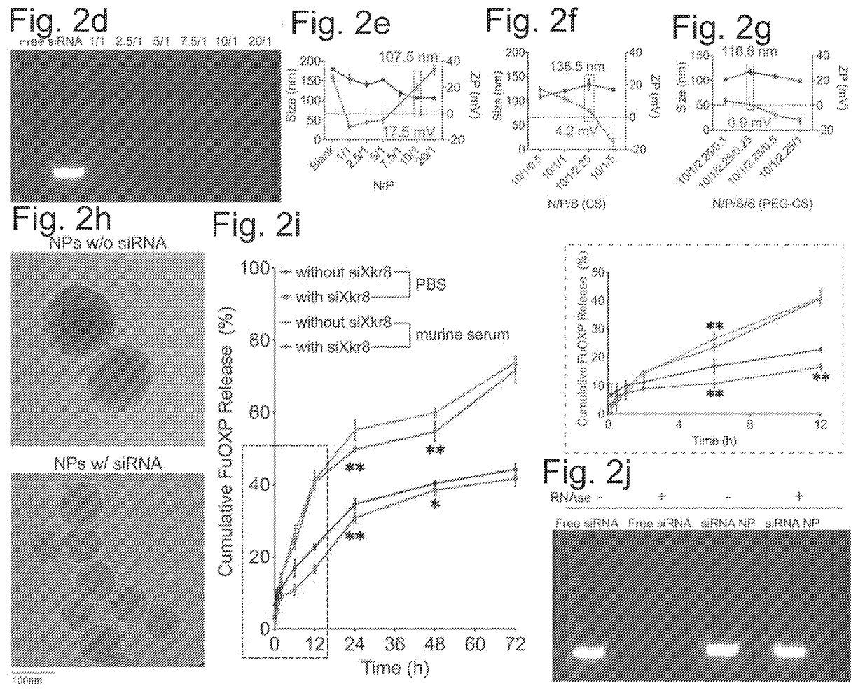

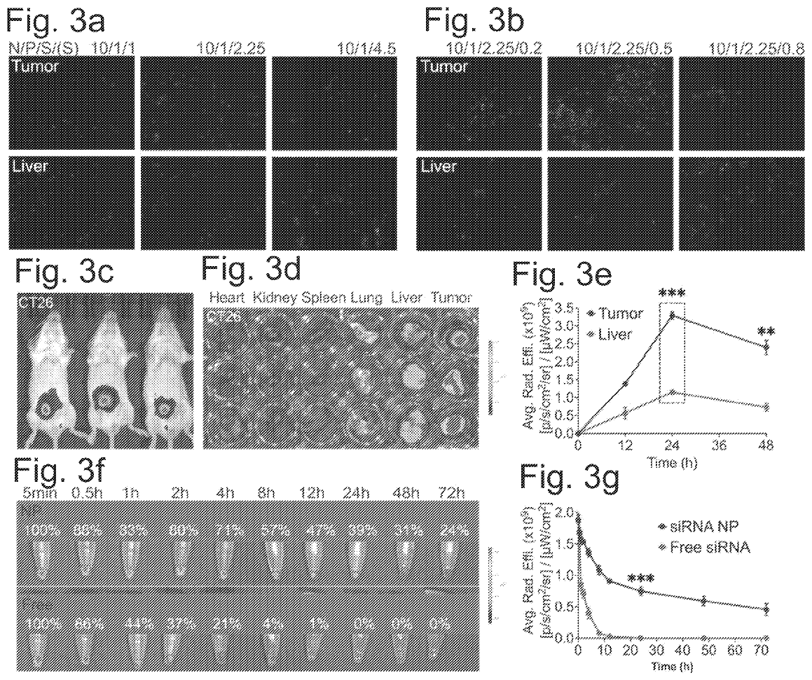

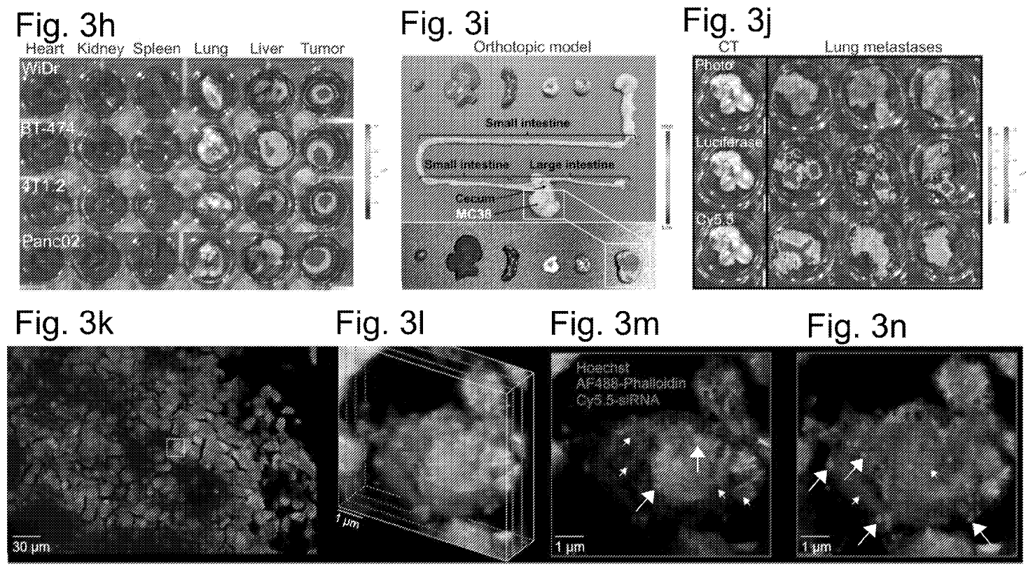

FIG. 2a illustrates the major components and steps in the development of the PMBOP-CP nanocarrier. PMBOP is an amphiphilic cationic polymer that self-assembles to form micelles in aqueous solutions. The 1-octadecene lipid motif in PMBOP polymer is expected to facilitate the interaction with cell membrane and improve transfection, while also helping to improve the loading of FuOXP into the hydrophobic/lipophilic core. The biguanidine motif was designed to enhance the interaction with siRNA as a result of its highly cationic nature. The synthesis route of PMBOP is shown in Scheme 1 (FIG. 3a) of PCT International Patent Application No. PCT/US22/43938 and its structure was confirmed by nuclear magnetic resonance (¹H NMR) spectroscopy.

PMBOP polymer had a low CMC of 0.0033 mg/mL (FIG. 2b) and readily formed micelles in PBS with a size of 173.2 nm. FuOXP could be loaded into PMBOP micelles at a carrier/drug weight ratio as low as 2/1 (FIG. 2c). Both drug-free and FuOXP-loaded PMBOP micelles readily formed complexes with siRNA. Gel retardation assay shows that siRNA was effectively incorporated into micelles at nitrogen/phosphate (N/P) ratios of 1 and above (FIG. 2d). At an N/P ratio of 10/1, the resulting PMBOP/FuOXP/siRNA complexes were positively charged (+17.5 mV) and 107.5 nm in size, smaller than PMBOP micelles loaded with FuOXP alone (179.1 nm)(FIG. 2e), suggesting that siRNA wrapped around and stabilized the micelles. Similar results were shown when siRNA formed complexes with drug-free PMBOP micelles.

Drug carriers with cationic surface are not suitable for systemic delivery to distant solid tumors. Therefore, the PMBOP/FuOXP/siRNA complexes formed at an N/P ratio of 10/1 were subjected to surface coating, application or modification with a mixture/application of chondroitin sulfate (CS) and PEG-CS to form PMBOP-CP nanoparticles (NPs). CS is a highly negatively charged molecule and can be used to decrease the surface positive charge of the resulting NPs. CS, like, for example, hyaluronic acid (HA) is also a natural ligand for CD44, which is overexpressed in various types of cancer cells and tumor endothelial cells (ECs). CS- and HA-based NPs have been studied extensively as carriers for tumor targeting. One barrier that limits the effectiveness of HA- or CS-mediated tumor targeting is the expression of CD44 on liver sinusoidal endothelial cells (LSECs) that, as a result of their abundance, rapidly remove most circulating NPs. Indeed, many reported HA- and CS-coated NPs showed extensive liver uptake with a level that is significantly higher than that in tumor. Therefore, small amount of PEG-CS were included with the expectation of improving the EPR effect by minimizing the “nonspecific” uptake by liver. As shown in FIG. 2f, increasing the amount of CS led to gradual neutralization of positive charges. At an N/P/sulphate (S) ratio of 10/1/2.25, the resulting NPs were 136.5 nm in size and slightly positive charged (+4.2 mV). Under this condition, the incorporation of small amount of PEG-CS (from a molar ratio of CS/PEG-CS of 2.5/0.1 to 2.25/0.25) led to further neutralization/shielding of positive charges and almost charge neutrality (FIG. 2g). Further increase in the amount of PEG-CS led to formation of negatively charged NPs (FIG. 2g). The morphology of PMBOP-CP NPs with and without loaded siRNA was examined by cryo-electron microscopy (cryoEM) revealing spherical particles of relatively uniform diameter in each case (FIG. 2h). FuOXP loaded into PMBOP-CP NPs showed a slow kinetics of release in PBS but the release became accelerated upon exposure to mouse serum (FIG. 2i). When complexed with siRNA, the drug release rates were slightly decreased in both PBS and mouse serum (FIG. 2i). Moreover, siRNA loaded into PMBOP-CP NPs was well protected from degradation by RNAse (FIG. 2j).

In general, any compound with suitable negative charge can be conjugated with PEG (and/or another hydrophilic polymer) to anchor the hydrophilic polymer conjugate to the nanostructure. Such a compound can, for example, be another negatively charged CD44 ligand, a bio-compound, a synthetic compound, etc. Alternatively, a portion of the hydrophilic polymer may be modified to include a negative charge.

A s.c. tumor model (CT26) was used for the initial optimization of PMBOP-CP NPs. The in vivo distribution of the Cy5.5-labeled siRNA in tumors and liver was examined by fluorescence microscopy at 24 h following i.v. injection of various PMBOP-CP NPs that were prepared at a N/P ratio of 10/1 and coated with various amounts of CS/PEG-CS, respectively. Increasing the N/P/S (CS) ratio from 10/1/1 to 10/1/2.5 was associated with a gradual increase of Cy5.5 signals in tumors and a concomitant decrease of signal in liver. Further increases in the amounts of CS resulted in decreases of the signals in tumor and increased signals in liver as determined via fluorescence microscopy (FIG. 3a). The tumor-targeting efficiency of CS-coated NPs was then studied to determine if the CS-coated NPs can be further improved via incorporation of PEG-CS. The N/P/S ratio was maintained at 10/1/2.25 while gradually increasing the amounts of PEG-CS. As illustrated in FIG. 3B, increasing the ratio of CS/PEG-CS from 2.25/0.2 to 2.25/0.5 led to further increases of Cy5.5 signals in tumors while the signals in liver were decreased as determined via fluorescence microscopy. Further increases in the amounts of CS-PEG resulted in decreased signals in tumors with increased signals in the liver (FIG. 3b). All subsequent studies were conducted with NPs prepared at a N/P/S(CS)/S(PEG-CS) ratio of 10/1/2.25/0.5.

FIGS. 3c-C illustrate NIR images at different times following i.v. injection of Cy5.5-siRNA NPs. Whole-body imaging showed that the Cy5.5 signals were concentrated in the tumor areas at 24 h (FIG. 3c). The ex vive imaging data (FIG. 3d) were consistent with the results of whole-body imaging. The levels of fluorescence signals in tumors were significantly higher than those in liver. Little signals were seen in heart, kidney, spleen, and lungs. FIG. 3e shows that the siRNA signal in tumors increase over time, peaked at 24 h and slowly declined thereafter. The siRNA NPs stayed in the blood significantly longer than free siRNA (FIGS. 3f and 3g).

Effective tumor targeting in several other s.c. tumor models including human colon cancer (WiDr), human breast cancer (BT-474), murine pancreatic cancer (Panc02), and murine breast cancer (4T1.2) were also demonstrated (FIG. 3h). A similar result was also observed in an orthotopic murine colon cancer model (FIG. 3i). A potential limitation of NPs is their low efficiency in targeting disseminated tumors and metastases as a result of the limited EPR in those small tumor lesions. Interestingly, a preliminary study showed that PMBOP-CP NPs effectively accumulated in metastatic tumors in the lung established by tail vein injection of CT26 tumor cells while minimal signal was seen in normal mouse lung (FIG. 3j). Further studies may be carried to define the efficiency of tumor targeting at different stages of lung metastasis and the underlying mechanism. FIG. 3k illustrates widespread distribution of Cy5.5 signal in tumor sections. At high magnifications, Cy5.5 signal was distributed in both punctuated (major) and diffused patterns, indicating that the majority of the endocytosed siRNA was entrapped in the endosome/lysosome while some was released into the cytosol (FIG. 3l-m). Colocalization of Hoechst and Cy5.5 was also observed, indicating that some siRNA accumulated in the nucleus following endosomal escape (FIG. 3l and FIG. 3n).

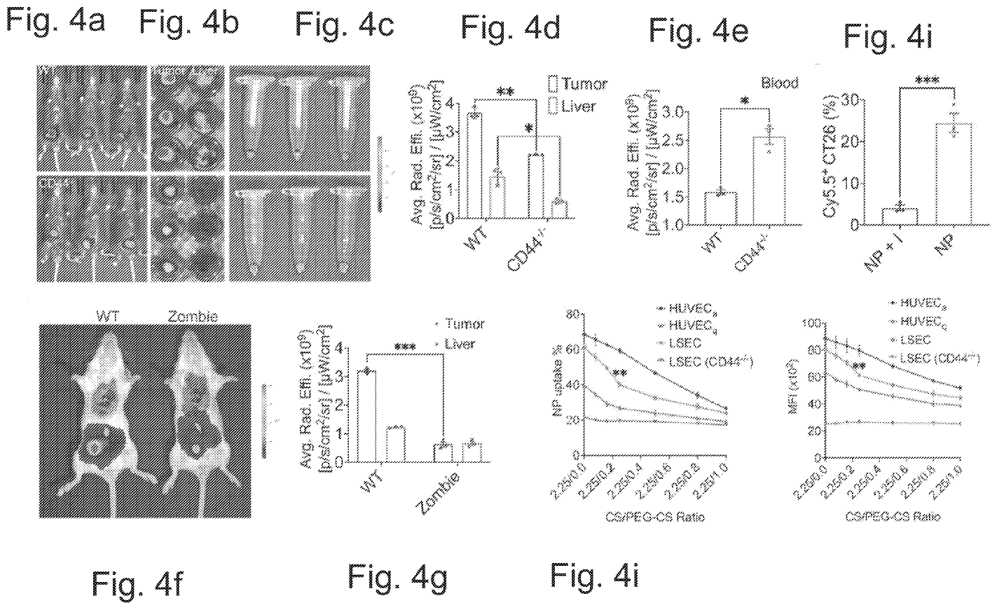

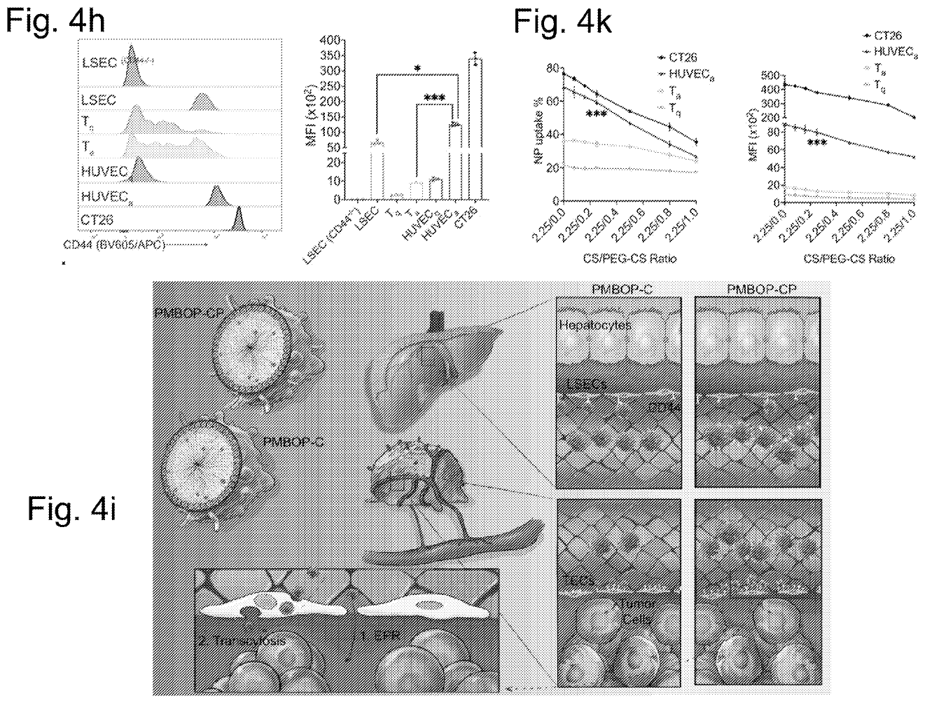

CD44-mediated transcytosis plays a role in tumor-targeting. In that regard, the unexpected decreases in tumor uptake that was associated with increased PEG shielding (PEG-CS/CS>0.5/2.25) indicates that CS-mediated active targeting likely plays a role in the overall tumor targeting. To examine whether the CD44-mediated ECs targeting plays a role, NIR imaging was similarly performed in CD44^{-/-} mice. As shown in FIGS. 4a and 4b, Cy5.5 siRNA NPs were highly effective in accumulating at tumor tissues in WT mice. However, the Cy5.5 signals in tumor tissues were decreased significantly in CD44^{-/-} mice. The uptake of Cy5.5 siRNA NPs was also decreased in the liver in CD44^{-/-} mice (FIGS. 4b and 4d). Interestingly, the Cy5.5 siRNA signal in blood was increased in CD44^{-/-} mice (FIGS. 4c and 4e), indicating that Cy5.5 siRNA NPs were highly stable in the blood for a significant period of time and that the CD44-mediated tumor ECs contributed to the overall tumor targeting. Meanwhile, CD44 in the LSECs may also contribute to the uptake of the NPs in the liver.

The tumor-targeting efficiency of PMBOP-CP NPs was also significantly decreased in Zombie mouse model (FIGS. 4f and 4g) in which the passive targeting mechanism such as EPR remains active while the active trans-endothelial transport is inhibited, indicating that both active and passive targeting mechanisms contribute to the overall tumor targeting by PMBOP-CP NPs. The Zombie mouse mode is, for example, discussed in Sindhwani, S. et al. The entry of nanoparticles into solid tumors. Nature Materials 19, 566-+, doi:10.1038% s41563-019-0566-2 (2020).

To further investigate the respective role of CD44 in tumor ECs and LSECs in interacting with PMBOP-CP NPs, the uptake of Cy5.5 siRNA NPs by primary mouse LSECs and human umbilical vein endothelial cells (HUVECs) was examined. HUVECs cultured in the absence of basic fibroblast growth factor (bFGF) are quiescent (HUVEC_{q}) and express low levels of CD44 while HUVECs cultured with bFGF become activated (HUVECₐ) and express a higher level of CD44, which are often used to model tumor ECs. FIG. 4h shows that the expression level of CD44 on HUVECₐ was about ˜1.9- and ˜13.5-fold higher than that on LSECs and HUVEC_{q} respectively. NPs coated with CS only (without PEG-CS) were effectively taken up by both HUVECₐ and LSECs with more NPs being taken up by HUVECₐ (69.3 vs 61.5%, P<0.01; FIG. 4i). It is also apparent that the HUVEC_{q} took up significantly less amounts of the NPs compared to HUVECₐ (FIG. 4i). Similar results were shown for CD44^{-/-} LSECs in comparison with WT LSECs, suggesting that CD44-mediated endocytosis likely plays a role in the cellular uptake of the CS-coated NPs by both tumor ECs and LSECs.

Incorporation of PEG-CS led to decreased cellular uptake of the NPs in a PEG dose-dependent manner in both HUVECₐ and LSECs. However, PEG-CS clearly showed more impact on the uptake by LSECs compared to HUVECₐ. At a ratio of CS/PET-CS of 2.25/0.25, the level of uptake by LSECs decreased to 39.8% but remained at 58.7% for HUVECₐ (FIG. 4i). At a ratio of 2.25/0.5, which was found to be optimal in the in vivo studies hereof, a significant difference was also observed between the two groups (32.4% vs 47.6%). At a ratio of CS/PEG-CS of 2.5/1.0, the levels of uptake of the PMBOP-CP NPs in both types of cells decreased to comparable levels (24.4% vs 26.8%). Similar results were obtained when the cellular uptake was evaluated by the average fluorescence intensity per cell (FIG. 4i).

CD44 has been shown to be capable of mediating transcytosis. To explore a potential role of transcytosis in tumor targeting, a co-culture experiment with HUVECₐ and CT26 cells using a Transwell plate was conducted. It was apparent that CT26 cells grown in the lower chamber were effectively transfected when Cy5.5.5-siRNA NPs were applied to HUVECₐ grown in upper chamber as determined by flow analysis of Cy5.5.5° CT 26 cells at 12h (FIG. 4j). This was significantly inhibited by dynasore, an endocytosis inhibitor, indicating the effectiveness of NPs hereof in mediating transcytosis through vascular ECs.

A concern is the expression of CD44 on immune cells, such as the activated T cells (Tₐ). However, their CD44 levels were significantly lower than those of tumor cells and “tumor ECs” (HUVECₐ)(FIG. 4h). Accordingly, the uptake of NPs by Tₐ was significantly less than that by tumor cells or tumor ECs in both the percentage of Cy5.5⁺ cells (FIG. 4k) and the average fluorescence intensity per cell (FIG. 4k). The uptake of NPs by Tₐ was further decreased in the presence of tumor cells, as the case in the tumor tissues in vivo.

The above data indicate that the active tumor-targeting of CS-based NPs can be “ironically” improved in vivo via shielding with appropriate amounts of PEG (FIG. 4l). CS-NPs without PEG shielding showed limited tumor targeting as a result of rapid removal of NPs by LSECs via CD-44-mediated uptake. Because of the relatively higher levels of CD44 on tumor ECs compared to LSECs, incorporation of “optimized” amounts of PEG will minimize the interaction of the NPs with LSECs without significantly compromising the binding to tumor ECs or tumor cells if the NPs manage to reach tumor cells via EPR (FIG. 4i). Use of excess amounts of PEG will block the interaction of CS-coated NPs with tumor ECs, which will result in the eventual uptake of NPs by Kupffer cells and possibly LSECs as well through CD44-independnet mechanism. The slight differences of optimal ratios in vitro and in vivo are likely attributed to, among other things, the subtle differences in the expression levels of CD44 between model tumor ECs and the tumor ECs in mice.

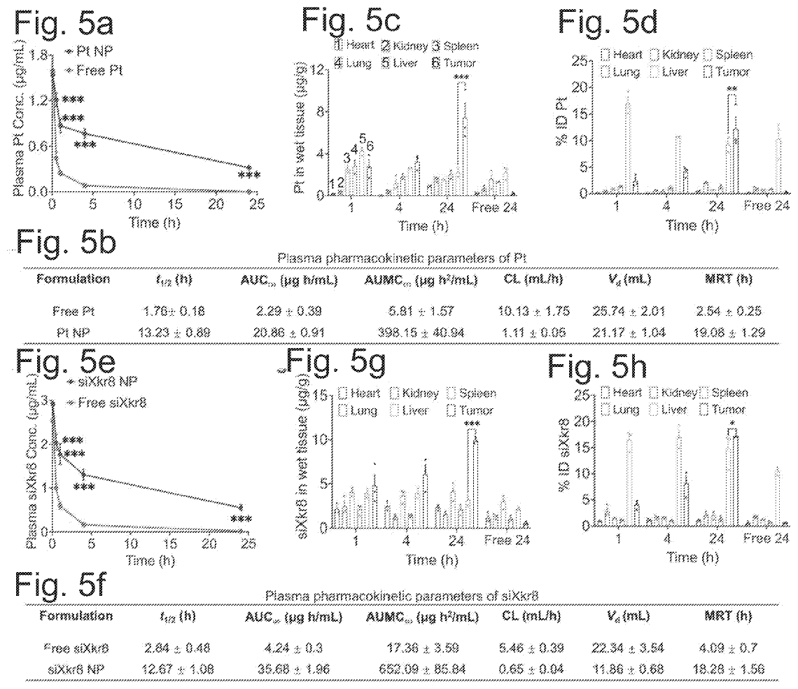

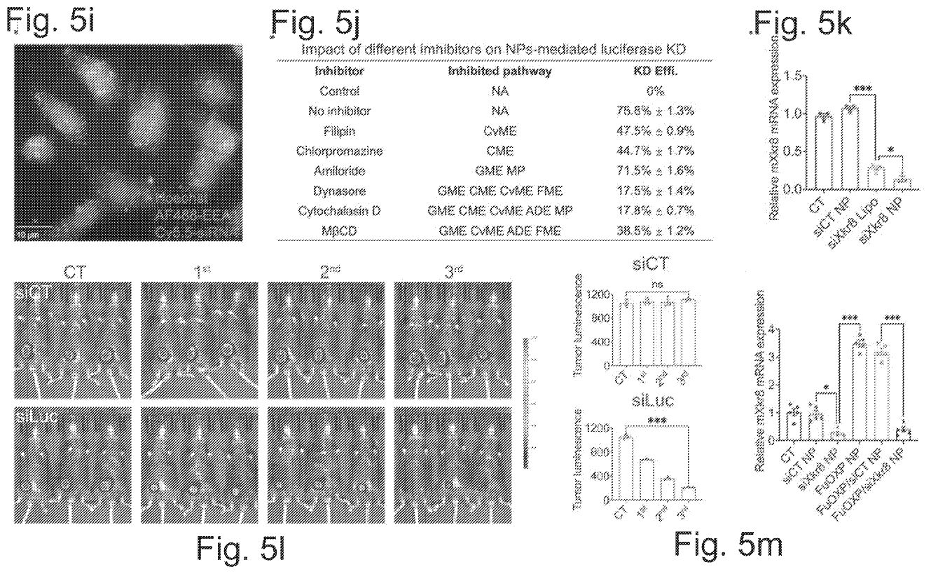

In vivo PK and tissue distribution of siXkr8 and FuOXP following i.v. administration of FuOXP/siXkr8 NPs, and the efficiency of gene knockdown were also studied. The characterization work described above led to the development of an “optimized” PMBOP-CP nanocarrier with well-defined biophysical properties for the studies hereof. The PK and tissue distributions of FuOXP and Cy5.5-siXkr8 that were co-loaded into the NPs at different times following i.v. injection were then studied. The fate of FuOXP was followed by directly quantifying the platinum (Pt) in blood and tissues without extraction using Inductively coupled plasma mass spectrometry (ICP-MS). The amount of siRNA was determined by qRT-PCR or fluorometer following extraction from blood and tissues. FIG. 5a shows the concentrations of Pt over time in blood following i.v. injection of free FuOXP/siXkr8 or FuOXP/siXkr8 NPs into tumor-free mice. The pharmacokinetic parameters were obtained by fitting the blood Pt concentration versus time using a non-compartmental model (FIG. 5b). FuOXP formulated in the NPs showed substantially greater t_{1/2}, AUC, and Cₘₐₓ while its V_{d} and CL were significantly lower than those of free FuOXP. The prolonged half-time of Pt in blood with FuOXP NPs was translated into significantly enhanced accumulation of Pt in the tumor tissues. The concentration of Pt in tumors increased over time following i.v. administration. At 24 h following injection, the Pt concentration in the tumors was about 3.3-fold higher than in liver (FIG. 5c) and the total amount of Pt in the tumor tissues was 12.2% of injected does (ID) (FIG. 5d). Pt was found largely in the liver and was barely detectable in tumors at 24 h following injection of free FuOXP (FIGS. 5c-d). Similar results were observed for the PK and tissue distribution of siXkr8 by either qRT-PCR (FIGS. 5e-b) or fluorescence measurement.

Following demonstration of enhanced delivery of siRNA to tumor tissues via PMBOP-CP NPs, the efficiency of siRNA-mediated knockdown of target genes was further examined in vitro and in vilo. As shown in FIG. 5i, Cy5.5-siRNA NPs were effectively taken up by cultured CT26 cells. Delivery of luciferase siRNA (siLUC) to MC38-Luc cells using PMBOP-CP NPs led to significantly decreased luciferase activity (FIG. 5j). The efficiency of silence was drastically attenuated when the cells were pretreated with filipin, chlorpromazine, amiloride, dynasore, cytochalasin D, or MPCD, respectively prior to transfection (FIG. 5j), suggesting the involvement of clathrin (CME)-, caveolin (CvME)-, flotillin (FME)- and Arf6 (ADE)-dependent endocytosis in siRNA delivery. FIG. 5k shows that the mRNA levels of Xkr8 were significantly decreased in cultured CT26 cells at 20 h following treatment with SiXkr8 NPs.

In vho delivery ofsiLuc NPs also led to gradual decreases in luciferase activity in MC38-Luc tumor-bearing mice upon repeated injections as assessed by whole-body bioluminescence imaging. The level of tumor luminescence was reduced by 34.3% after the first treatment and 80.1% following three treatments (FIG. 5l). FIG. 5m shows that SiXkr8 NPs effectively inhibited both basal and FuOXP-induced Xkr8 mRNA levels in vivo.

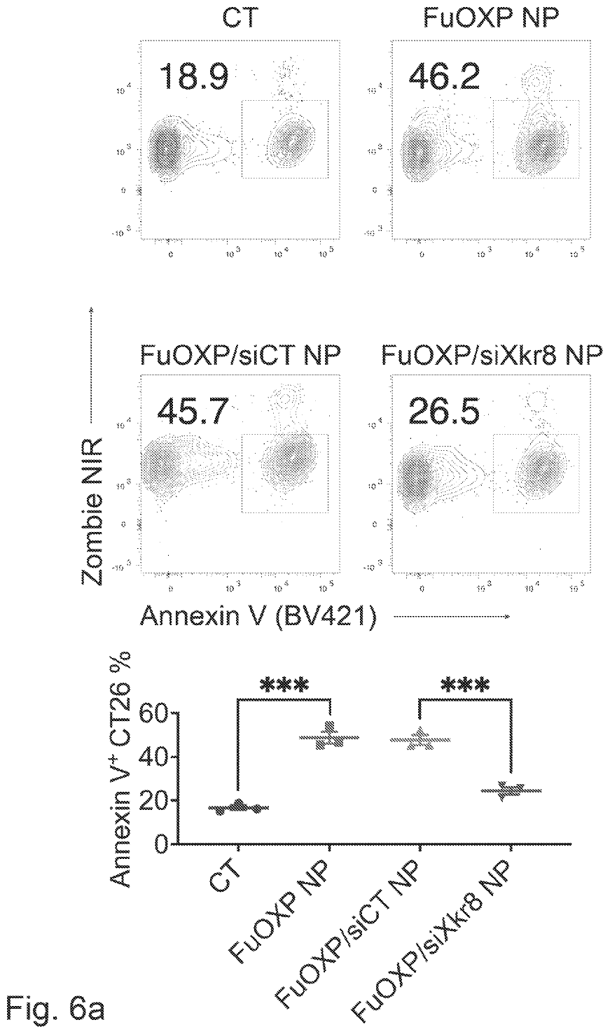

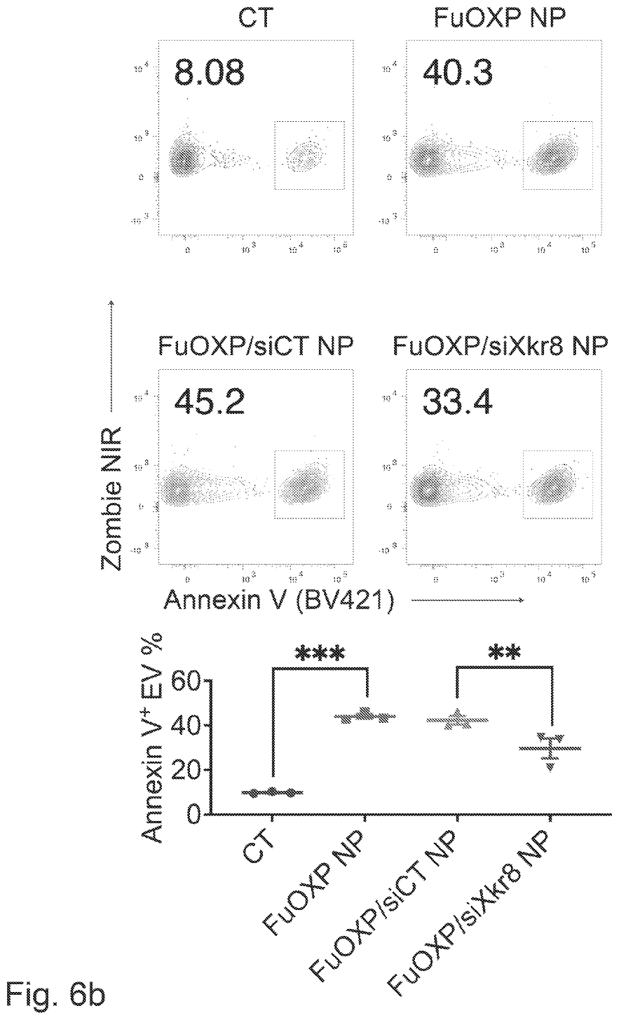

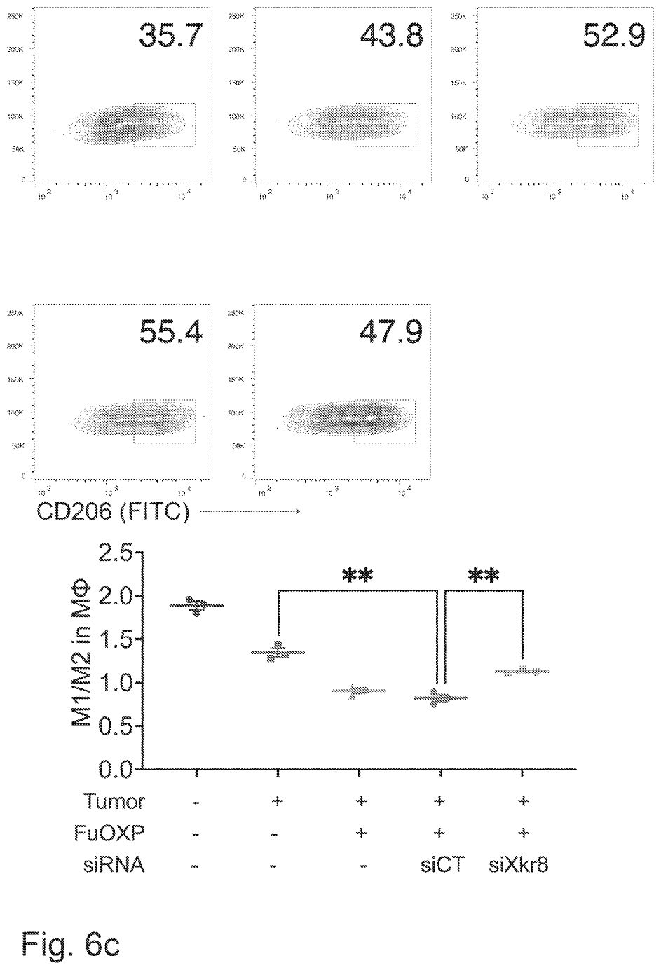

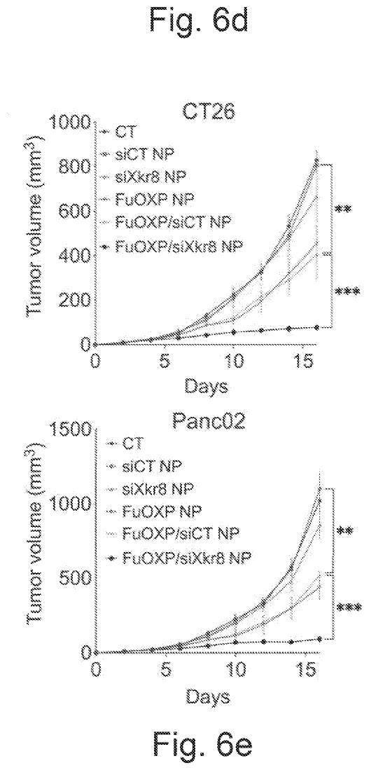

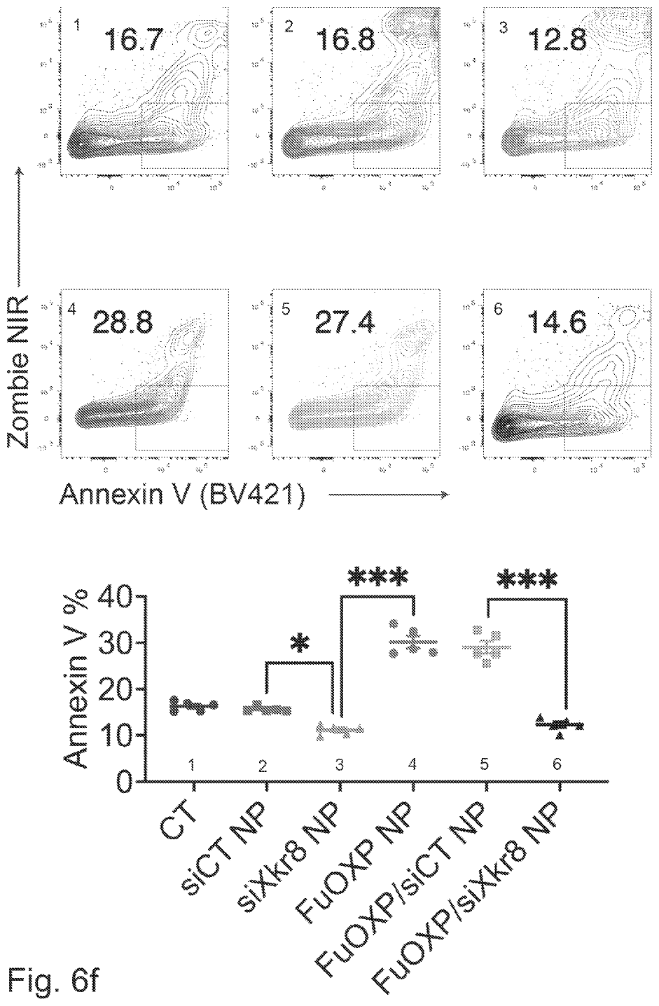

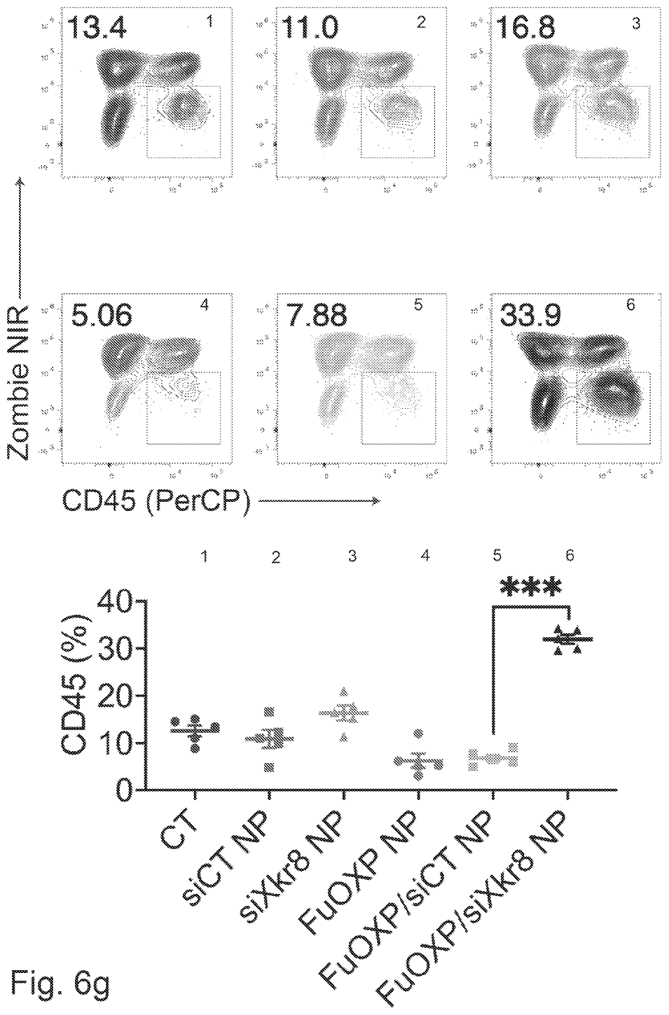

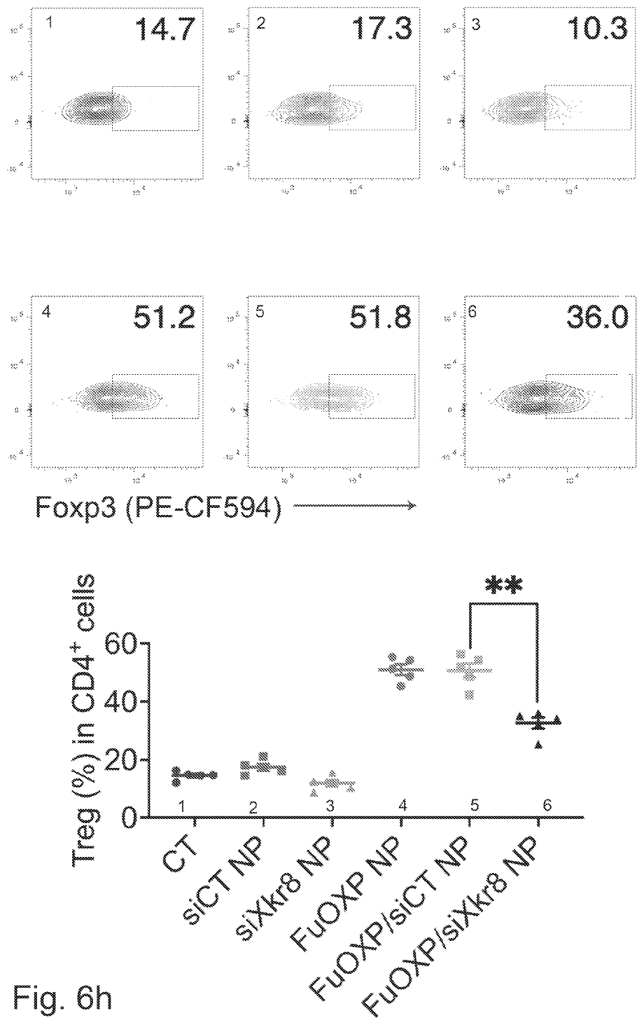

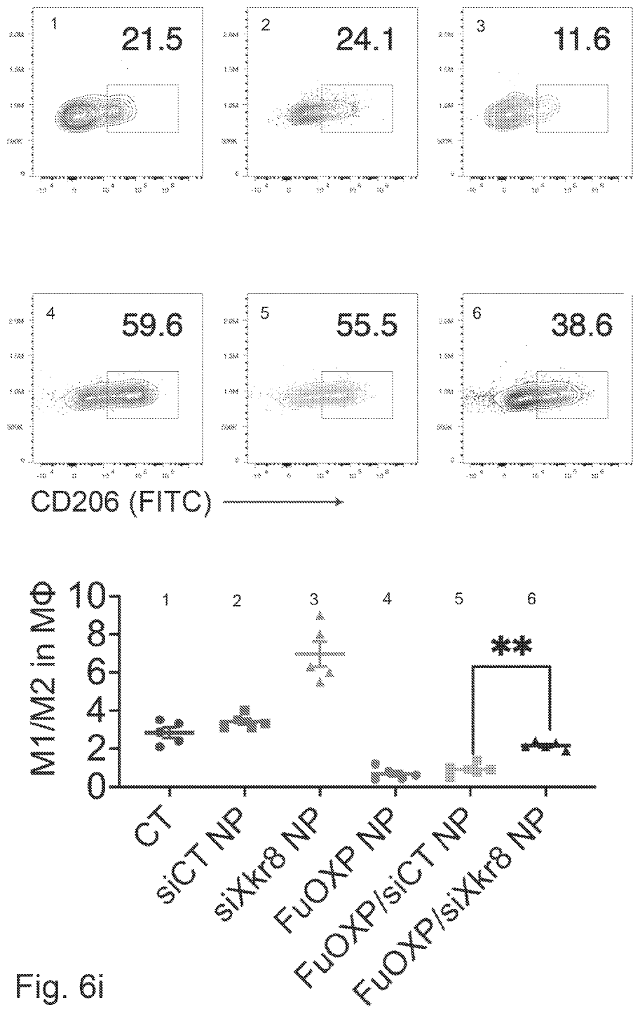

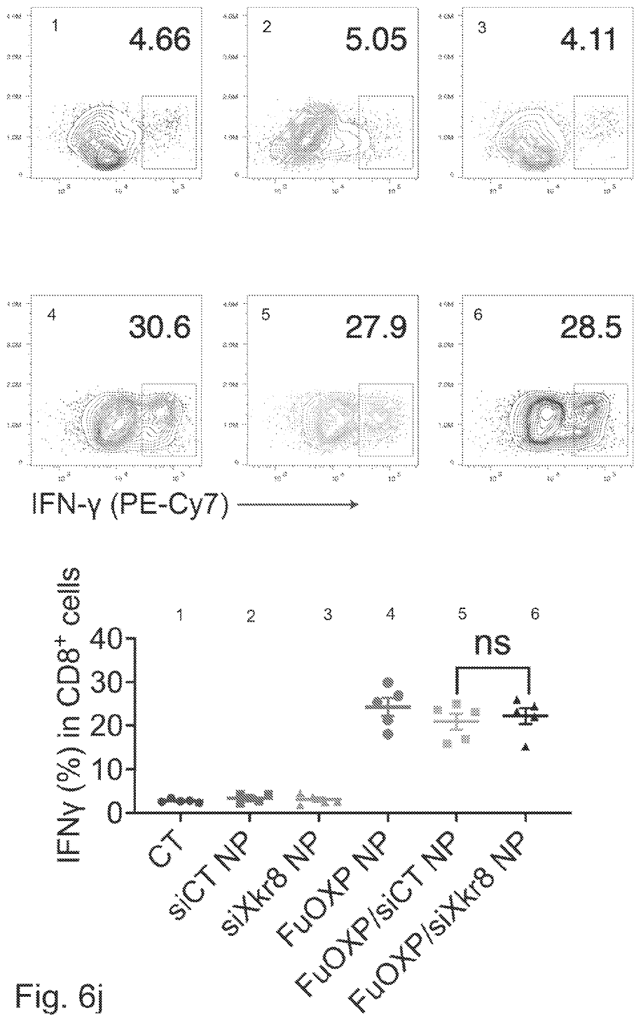

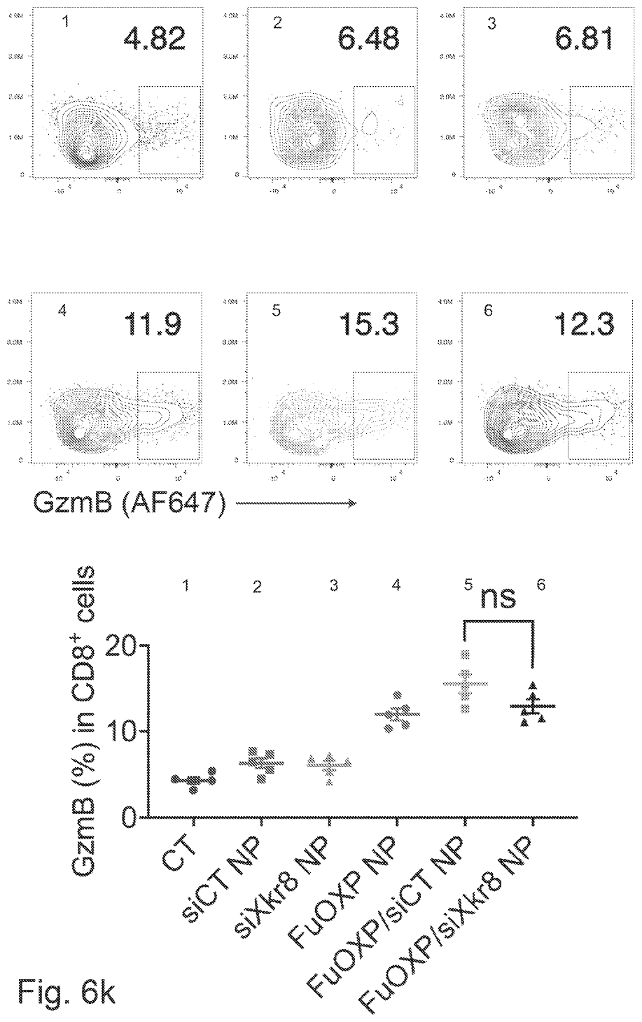

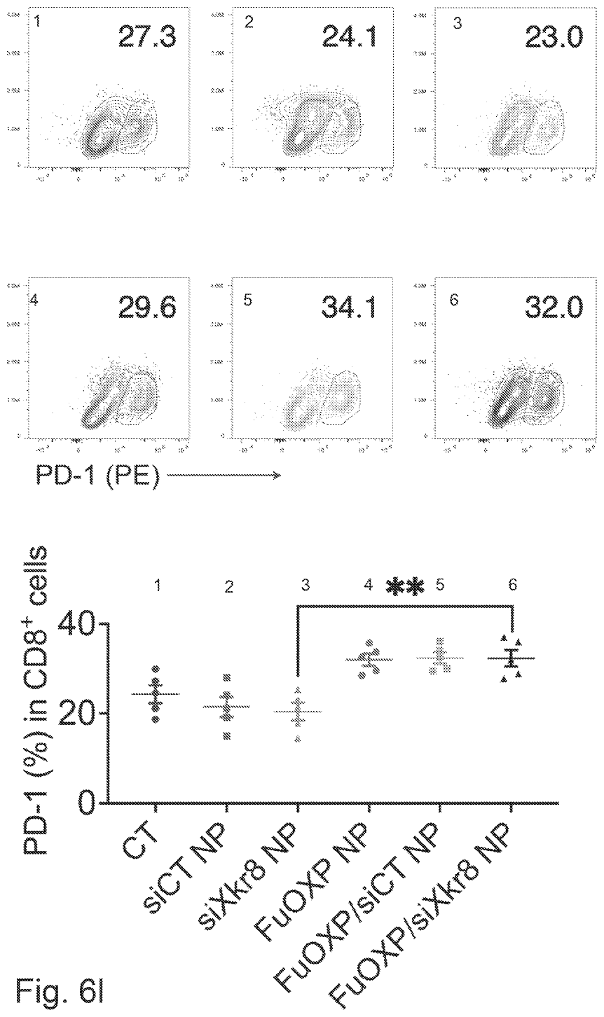

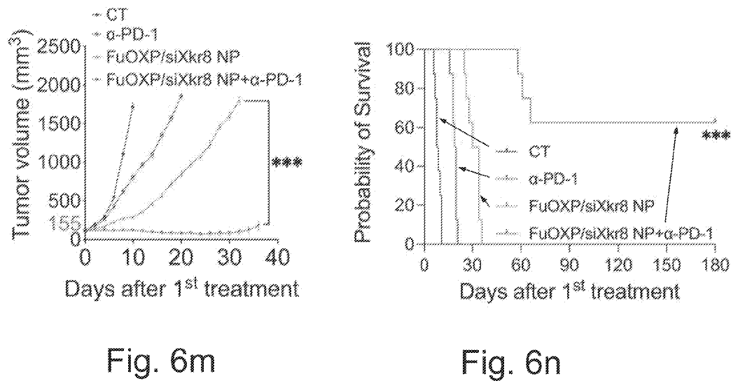

Consistent with the literature studies (see Song, W. el al. Synergistic and low adverse effect cancer immunotherapy by immunogenic chemotherapy and locally expressed PD-LI trap. Nat Commun 9, 2237, doi:10.1038/s41467-018-04605-x (2018)), treatment of CT26 tumor cells with FuOXP NPs led to significant increases in the level of surface PS (FIG. 6A). This increase was almost abolished when the cancer cells were pre- and then co-treated with SiXkr8. Pretreatment with SiXkr8 NPs also partially abolished the FuOXP-induced increases in the amounts of the PS-positive extracellular vesicles (EVs) (FIG. 6b) that have also been reported to be highly immunosuppressive.

FIG. 6C shows that co-culture of primary mouse macrophages with CT26 cells pretreated with FuOXP/control siRNA (SiCT) NPs resulted in decreases in M1/M2-like ratios. These changes were significantly reversed when CT26 cells were first treated with SiXkr8 followed by FuOXP/SiXkr8 cotreatment.

The above data prompted investigation of the therapeutic potential of codelivery of FuOXP and SiXkrB using PMBOP-CP NPs in CT26 and Panc02 models. The two models were chosen as 5-FU and oxaliplatin were important treatments for both colon and pancreatic cancers, but with limited efficacy. SiXkr8 NPs alone slightly inhibited the growth of CT26 tumor (FIG. 6d). This result is likely due to the specific Xkr8 knockdown as a CT26 subline with stable Xkr8 knockdown showed significant delay of tumor growth in immunocompetent mice. FuOXP NPs alone showed modest effect in controlling the tumor growth (FIG. 6D). However, the combination of both led to significant improvement in the antitumor activity (FIG. 6D). Similar results were observed in Panc02 pancreatic cancer model (FIG. 6E).

The frequency of PS-positive cells was reduced following treatment with SiXkr8 NPs alone (FIG. 6F). Treatment with FuOXP NPs led to drastic increases in the frequency of PS-positive cells. This increase was almost abolished when FuOXP and SiXkr8 were co-delivered to tumors via PMBOP-CP NPs (FIG. 6F).

Treatment with FuOXP/SiXkr8-coloaded NPs led to significant increases in the numbers of CD45⁺ cells, IFNγ⁺ CD8⁺ T cells, and GzmB⁺ CD8⁺ T cells as well as a decrease in the numbers of CD4⁺ Treg cells compared to control or the group treated with FuOXP NPs alone (FIGS. 6G-6K). Treatment with PMBOP-CP NPs loaded with FuOXP alone or co-loaded with FuOXP and SiCT led to significant decreases in the M1/M2-lie ratio (FIG. 6l). These decreases were almost completely reversed in the group treated with FuOXP/SiXkr8-coloaded NPs (FIG. 6l). Similar flow data were observed in Panc02 tumor model.

FIG. 6l shows that FuOXP NPs with or without co-loaded siXkr8 also caused significant upregulation of PD-1 expression in CD8⁺ T cells in the Panc02 model, suggesting potential for a combination therapy with anti-PD-1 antibody. Indeed, combination of FuOXP/siXkr8 NPs with anti-PD-1 led to a drastic improvement in therapeutic efficacy as evident from significant inhibition of tumor growth (FIG. 6m) and prolongation of survival time (FIG. 6n).

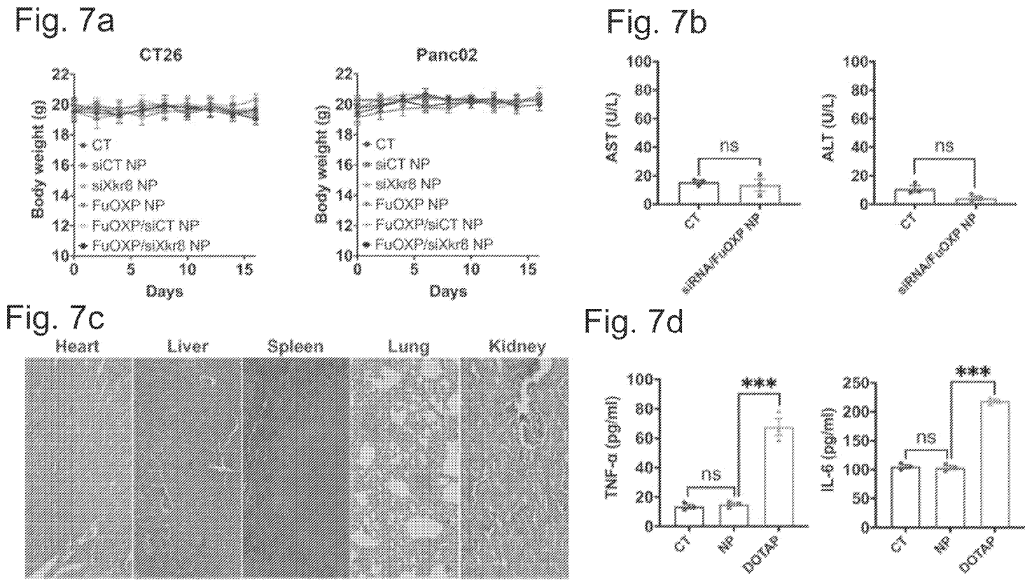

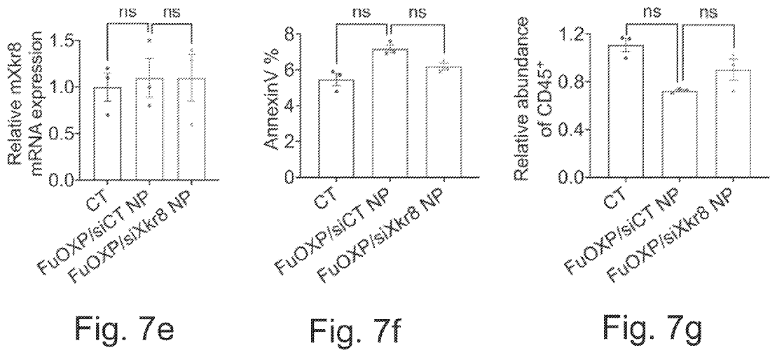

FuOXP/siRNA-coloaded NPs were well tolerated at the doses used as shown by normal body weight gains (FIG. 7a), minimal changes in the serum levels of ALT and AST (FIG. 9b), and normal histology of several major organs examined (FIG. 7c). In addition, unlike siRNA complexed with cationic DOTAP liposomes that induced significant increases in serum levels of TNF-α and IL-6, siRNA PMBOP-CP NPs did not affect the serum levels of two proinflammatory cytokines (FIG. 7d). FuOXP NPs also showed minimal impact on the level of mXkr8 mRNA (FIG. 7e) as well as the number of PS⁺ cells (FIG. 7f) in liver at the dose used in therapy study. In addition, there were no obvious change in the number of CD45⁺ cells following treatment with FuOXP/siXkr8 NPs (FIG. 7g).

In summary, it has been shown that Xkr8 expression in tumor cells was significantly induced by chemotherapeutic agents. In addition, the hypothesis-driven studies hereof led to the development of a representative PMBOP-CP-based nanocarrier that is highly effective in tumor targeting through effective tumor ECs-mediated active targeting while minimizing the LSECs-mediated liver uptake. In addition to enhanced delivery of both types of therapeutics to tumors, the strategies hereof have the advantage of selectively delivering SiXkr8 to those tumor cells that are exposed to chemodrugs. Therefore, the codelivery formulations and methodologies hereof are particularly effective in antagonizing the Xkr8 mRNA that is induced in situ by co-delivered chemotherapeutic drug. Codelivery of SiXkr8 and FuOXP led to significant improvement in tumor immune microenvironment and enhanced antitumor activity. It is interesting to note that Xkr8^{-/-} mice are developmentally normal, and do not develop autoimmunity in CS7BL/6 mice, indicating that targeting Xkr8 in tumor cells is a safe approach. Safety concerns with Xkr8 targeting can be further minimized through tumor-targeted delivery using NPs. Targeting Xkr8 in combination with chemotherapy may represent a novel and effective immunochemotherapy for the treatment of various types of cancers including, for example, colon and pancreatic cancers.

### Experimental Examples

Materials. Dulbecco's Modified Eagle's Medium (DMEM), Poly(maleic anhydride-alt-1-octadecene) (PMAO, Mn=30,000-50,000), ethylenediamine, DMF, tert-butanol and trypsin-EDTA solution were purchased from Sigma-Aldrich (MO, USA). PEGmK-NHS was purchased from JenKem Technology (TX, USA). Dicyandiamide was purchased from TCI America (OR, USA). Fetal bovine serum (FBS) and penicillin-streptomycin solution were purchased from Invitrogen (NY, USA). Antibodies used for flow cytometry were purchased from established vendors such as BioLegend and BD Biosciences. Dicyandiamide was purchased from TCI America Company (PA, USA).

Xkr8 siRNA and other oligonucleotides: Murine Xkr8 siRNA (siXkr8) and control (non-targeting) siRNA (siCT) were designed and synthesized by Ambion (TX, USA). Cy5.5-siXkr8 was synthesized by Sigma-Aldrich (MO, USA). Cy5.5 was introduced to siXkr8 via phosphate linkage using phosphoramidite chemistry. Primers for RT-PCR amplification of mXkr8 and hXkr8 mRNAs, and siXkr8 were provided by IDT (IA, USA). The sequences of siXkr8, siCT, and other primers are shown in Table. 1. All siRNA sequences are non-coding RNA and were decorated with deoxythymidine dinucleotide tt and tc overhangs at the 3′ end to increase complex stability and enhance protection from RNase and yield better gene silencing. Overhangs are not part of the sequence, nor base pairs. In vivo siRNA was quantified using an established method involving two steps as described, for example, in Raymond CK, Roberts BS, Garrett-Engele P, Lim L P, Johnson J M. Simple, quantitative primer-extension PCR assay for direct monitoring of microRNAs and short-interfering RNAs. RNA. 2005 November; 11(11):1737-44. doi: 10.1261/rna.2148705. PMID: 16244135; PMCID: PMCI370860. In the first step, a tailed, gene-specific primer (GS primer) was used to convert the RNA template into cDNA. A “universal” PCR binding site was introduced to one end of the cDNA molecule to extend the length of the cDNA to facilitate subsequent monitoring by qPCR. In the second step, the resulting primer-extended, full-length cDNA was quantified by real-time PCR using a combination of a locked nucleic acids (LNA)-containing, which is to restrict the flexibility of the ribofuranose ring and lock the structure into a more rigid bicyclic formation (followed by +), siRNA-specific “reverse” primer (LNA-R primer) and a generic universal primer.

TABLE 1

| Seq | | | | | |
|---|---|---|---|---|---|
| ID | | Mol | | | Sequence |
| No | Description | Type | Organism | Name | 5′→3′ |
| 1 | Murine siRNA | RNA | (mus musculus) | siXkr8 (m) | ggcacaaattttatgcctg |
| | Sense | siRNA | | sense | |
| | | | | siRNA ID #: 164623 | |
| 2 | Murine siRNA | RNA | (mus musculus) | siXkr8 (m) | caggcataaaatttgtgcc |
| | Antisense | siRNA | | antisense | |
| | | | | siRNA ID #: 164623 | |
| 3 | Human siRNA | RNA | (Homo | sixkr8 (h) | cccatttagcacagaagtt |
| | Sense | siRNA | Sapiens) | sense | |
| | | | | siRNA ID #: 123026 | |
| 4 | Human siRNA | RNA | (Homo | sixkr8 (h) | aacttctgtgctaaatggg |
| | Antisense | siRNA | Sapiens) | antisense | |
| | | | | siRNA ID #: 123026 | |
| 5 | Control siRNA | RNA | (artificial/ | siCT | caatattgcagctattcgg |
| | Sense | siRNA | Synthetic | sense | |
| | | | construct) | | |
| 6 | Control siRNA | RNA | (artificial/ | siCT | ccgaatagctgcaatattg |
| | Antisense | siRNA | Synthetic | antisense | |
| | | | construct) | | |
| 7 | PCR | DNA | (artificial/ | mXkr8 primer | gcactgctggattaccatcg |
| | Primer for | Other | Synthetic | forward | |
| | murine mRNA | | construct) | | |
| 8 | PCR Primer for | DNA | (artificial/ | mXkr8 primer | gccacatagtaggggaagacag |
| | murine | Other | Synthetic | rearward | |
| | mRNA | | construct) | | |
| 9 | PCR Primer for | DNA | (artificial/ | hXkr8 primer | cactgctcgactaccaccg |
| | human | Other | Synthetic | forward | |
| | mRNA | | construct) | | |
| 10 | PCR Primer for | DNA | (artificial/ | hXkr8 primer | ccacaggtagtagatcacggag |
| | human | Others | Synthetic | rearward | |
| | mRNA | | construct) | | |
| 11 | Gene-specific | DNA | (artificial/ | siXkr8/ | catgaacagctgggccaagaggcacaaatttt |
| | PCR primer for | Other | synthetic) | GS primer | |
| | murine siXkr8 | | | | |
| 12 | LNA-containing | DNA | (artificial/ | siXkr8/ | c+agg+cataaaatttgt |
| | PCR reverse | Other | synthetic) | LNA-R primer | |
| | primer | | | | |
| 13 | Universal PCR | DNA | (artificial/ | siXkr8/ | catgatcagctgggccaaga |
| | primer | Other | synthetic | universal primer | |

RNA: a = adenine, c = cytosine, g = guanine, t = uracil
DNA: a - adenine, = cytosine, g = guanine, t = thymine, “+” indicates “locked”
restriction/modification of the flexibility of the ribofuranose ring in the nucleobase preceding the “+” in which the ribose moiety is modified with an extra bridge connecting the 2′ oxygen and 4′ carbon

Mice: C57BL/6, BALB/c, NOD.Cg-Prkdc^{scid} I12rg^{mlWji}/SzJ (NSG) and B6.129(Cg)-Cd44^{tm1Hbg}/J (CD44^{-/-}) mice aged between 4-6 weeks were purchased from The Jackson Laboratories (CT, USA). Mice were housed under pathogen-free conditions according to AAALAC (Association for Assessment and Accreditation of Laboratory Animal Care) guidelines. The mouse-related experiments were performed in full compliance with institutional guidelines and approved by the Animal Use and Care Administrative Advisory Committee at the University of Pittsburgh. Mice were housed at an ambient temperature of 22° C. (22-24° C.) and humidity of 45%, with a 14/10 day/night cycle (on at 6:00, off at 20:00), and allowed access to food ad libitum.

Tumor cell lines: CT26 and MC38 murine CRC cell lines, HT29 and WiDr human CRC cell lines, Panc02 murine PCa cell line, PANC-1 human PCa cell line, 4T1.2 murine BCa cell line, and BT-474 human BCa cell line were obtained from ATCC (VA, USA). CT26 cells were cultured in RPMI-1640 medium supplemented with 10% FBS and penicillin/streptomycin (100 U/mL). MC38, Panc02, PANC-1, 4T1.2, and BT-474 cells were cultured in DMEM medium supplemented with 10% FBS and penicillin/streptomycin (100 U/mL). HT29 cells were cultured in McCoy's 5A medium supplemented with 10% FBS and penicillin/streptomycin (100 U/mL). WiDr cells were cultured in EMEM medium supplemented with 10% FBS and penicillin/streptomycin (100 U/mL). The cells were all cultured at 37° C. in a humidified atmosphere with 5% CO₂. For establishment of mouse tumor models, tumor cells between passages 3-10 in 100 μL of saline were subcutaneously (s.c.) inoculated into the right lower abdomen using a 27^{1/2} G needle.

Bulk RNA-seq analysis: RNA-seq was performed by the Health Sciences Sequencing Core at Children's Hospital of Pittsburgh. BALB/c mice (n=3) bearing s.c. CT26 tumors (˜200 mm³) received i.v. injection of FuOXP NPs (10 mg FuOXP/kg) with PBS as a control once every five days for three times. Tumors were harvested at 24 h after the last treatment. RNA-seq libraries were sequenced as 75-base paired-end reads at a depth of ˜73 to 77 million reads per sample. Reads were mapped to the mouse genome (GRCm38) using STAR Aligner 2.6.1a. Dobin, A. et al. STAR: ultrafast universal RNA-seq aligner. Bioinformatics 29, 15-21, doi:10.1093/bioinformatics/bts635 (2013). Gene expression quantification and differential expression analysis between control and FuOXP NPs treatment were performed using Cuffdiff of Cufflinks 2.2.1. Trapnell, C. et al. Differential analysis of gene regulation at transcript resolution with RNA-seq. Nat Biotechnol 31, 46-53, doi:10.1038/nbt.2450 (2013). Volcano plots were generated to show the overall differential expression, where the x axis indicates the log₂(fold change)(log₂FC) between FuOXP NPs and PBS and the y axis indicates the corresponding −log₁₀(P value).

Analysis of Xkr8 mRNA level by qRT-PCR: Groups of 5 BALB/c mice bearing s.c. CT26 tumors (˜200 mm³) received FuOXP NPs, empty NPs or PBS as described above and tumors were collected one day after the last treatment and subjected to qRT-PCR analysis as detailed below.

To examine the effect of different chemotherapeutic agents on the expression level of Xkr8 mRNA, CT26, Panc02, PANC-1 or HT29 cells were treated with various concentrations of FuOXP, DOX or PTX. Twenty-four h later, cells were collected and subjected to qRT-PCR of mXkr8 or hXkr8 as detailed below.

To examine the impact of ActD treatment on the expression level of Xkr8 mRNA, CT26 or PANC-1 tumor cells were treated with FuOXP (10 μM) for 12 h followed by addition of ActD (2 μM). At 2 or 4 h following ActD treatment, cells were collected and subjected to qRT-PCR of Xkr8 mRNA as described below. In another study, cells were treated with NAC (2.5 mM) for 12 h followed by treatment with FuOXP, DOX or PTX for another 12 h. qRT-PCR of Xkr8 mRNA was then similarly performed.

Tumor tissues or cells collected from the above experiments were subjected to RNA extraction by TRIzol™. cDNA was generated from the purified RNA using QuantiTect Reverse Transcription Kit (Qiagen, MD, USA) according to the manufacturer's instructions. Quantitative real-time PCR was performed using SYBR Green Mix on a 7900 HT PCR instrument (Applied Biosystems, MA, USA). Relative target mRNA levels were analyzed using delta-delta-Ct calculations and normalized to GAPDH. The primer sequences are shown in Table 1.

Analysis of hXkr8 protein level by Western blot: Cultured HT29 or PANC-1 cells received similar treatments as described above. Cells were then lysed with RIPA lysis buffer (Thermo Fisher Scientific, MA, USA) by gently shaking on ice for 30 min. After centrifugation at 12,300 RPM for 10 min, the supernatants were collected, and the concentrations of proteins were measured using Pierce BCA Protein Assay Kit (ThermoFisher Scientific, MA, USA). The protein samples were denatured by boiling for 5 min and loaded onto 10% SDS-PAGE gel for electrophoresis. The proteins in the gels were subsequently transferred onto PVDF membranes (Bio-Rad, CA, USA). The membranes were then incubated in blocking buffer (5% non-fat dry milk in TBST) for 1 h at RT, followed by incubation with anti-hXkr8 polyclonal antibody (ThermoFisher Scientific, MA, USA) in antibody dilution buffer (5% BSA in TBST, 1/2000 dilution) with gentle agitation overnight at 4° C. After washing with TBST for three times, the membranes were subsequently incubated with the secondary HRP-linked goat anti-rabbit IgG antibody (Cell Signaling Technology, MA, USA) for 1 h at RT. After another three washes with TBST, the membranes were incubated with Pierce™ ECL Western Blotting Substrate (ThermoFisher Scientific, MA, USA) for 1 min. Protein expression was normalized against β-Tubulin expression.

Synthesis of PMBOP: Poly(maleic anhydride-alt-1-octadecene) (PMAO, Mn=30,000-50,000, compound 1, 7 g, 20 mmol of repeating units) and dry and degassed DMSO(150 mL) were added into a 250 mL glass bottle equipped with magnetic bar and placed under an atmosphere of nitrogen. A volume of 6.67 mL ethylenediamine (100 mmol) in 50 mL dry and degassed DMSO solution was then added to the solution. After stirring at 160° C. under nitrogen for 48 hours, the solution was cooled down to room temperature, to which 1 L of HCl solution (2 mmol) was added. The precipitate was filtered and washed 3 times with water, and then dried under vacuum at 50° C. to obtain Poly(maleimideethylamine-alt-1-octadecene) polymer (PMO, compound 2). Then, 392 mg of compound 2(1 mmol of repeating units), 200 mg of PEG2K-NHS (0.1 mmol), 10 mL of dry DMSO and 1 mL TEA (triethylamine) were added into a 50 mL bottle equipped with magnetic bar. The solution was allowed to stir for 48 hours at room temperature. After the reaction, the solution was transferred to dialysis bag (MWCO 12,000-14,000) and dialyzed against water for 24 hours. After dialysis, the solution was filtered by P5 filter paper and lyophilized to obtain PEG-conjugated PMAO polymer with a yield of about 10-20%. PEG-conjugated PMAO polymer (100 mg) and dicyandiamide (840 mg, 10 mmol) were then dissolved in 10 mL of/ert-BuOH and refluxed with stirring for 12 hours. After the reaction, the solution was transferred to a dialysis bag (MWCO 12,000-14,000) and dialyzed for 24 hours against water. After lyophilization, the Poly(maleimideethylbiscarboximidamide-alt-1-octadecene)-Poly(maleimideethylpolyethylene-glycol-alt-1-octadecene) (PMBOP, compound 3) was obtained with a yield of ˜98%.

Preparation of FuOXP/siRNA-coloaded PMBOP NPs: PMBOP polymer (5 mg) and FuOXP (0.5 mg) of 10:1 ratio (w/w) were dissolved in DMSO and added to water with 15 times of the initial volume. The mixture was then transferred to a Amicon® Ultra 3K (3,000 MWCO) centrifugal filter device (Sigma-Aldrich, MO, USA). The device was centrifuged at 4,500 RPM for 15 min followed by the addition of 1 mL water. This step was repeated 3 times to remove any residual DMSO and concentrate the FuOXP-loaded micelles to a desired volume. Greater than 99% of PMBOP monomer was found to be incorporated into micelles based on the quantification of free FITC-labeled PMBOP following the filtration of FITC-labeled PMBOP micelles through a membrane of 100 nm pore size. SiXkr8 or siCT (0.1 mg/mL) was then mixed with FuOXP-loaded micelles at a 10:1 N:P ratio at RT for 20 min to form PMBOP/FuOXP/siXkr8 or PMBOP/FuOXP/siCT complexes. Subsequent incubation with a mixture of CS/PEG-CS of various ratios at RT for 20 min led to the formation of S/PEG-CS-decorated, FuOXP/siXkr8 co-loaded PMBOP-CP NPs. CS was found to be quantitatively (99.12%) incorporated into PMBOP-CP NPs under the “optimal” condition using FITC-labeled CS. The particle size (zeta average), zeta potential and polydispersity index (PDI) were measured by a Zetasizer from three batches of formulation. Drug loading capacity (DLC) and drug loading efficiency (DLE) of FuOXP were determined by high-performance liquid chromatography (HPLC). The siRNA concentration in the NPs was determined by Ribogreen assay after the NPs were disrupted by adding SDS (0.05%) and greater than 99% of siRNA was also found to be incorporated into the PMBOP-CP NPs. Complexation of siRNA with PMBOP polymer was confirmed by gel retardation assay. To examine the resistance of FuOXP/siXkr8 co-loaded NPs against nuclease-mediated degradation, the siRNA NPs (0.1 mg/mL siRNA, 0.35 mg/mL PMBOP) were incubated with RNase (50 U/mL) (NEB, MA, U.S.A) at 37° C. One h later, the NPs were disrupted by 0.05% SDS and the integrity of siRNA was examined by electrophoresis. Free siRNA was used as a control. The abbreviated empty NPs in this communication refer to PMBOP micelles coated with CS/PEG-CS while FuOXP NPs refer to FuOXP-loaded PMBOP micelles coated with CS/PEG-CS. FuXOP/siRNA NPs refer to PMBOP/FuOXP/siRNA complexes coated with CS/PEG-CS.

Cryo-electron microscopy: Samples were first checked with negative stain electron microscopy by applying 3 μL to a freshly glow-discharged continuous carbon on a copper grid and staining with a 1% uranyl acetate solution. Grids were inserted into a Thermofisher TF20 electron microscope (Thermofisher Scientific, MA, USA) equipped with a field emission gun and imaged on a TVIPS XF416 CMOS camera (TVIPS GmbH, Gauting, Germany) to visualize nanoparticle uniformity and concentration. Cryo-grids were prepared by pipetting 3 μL of sample on a Protochips C-flat CF-2/1-3CU-T grid (Protochips, NC, USA) that had been glow discharged at 25 mA for 30 s using an Emitech KX100 glow discharger. Grids were mounted in a Thermofisher Vitrobot Mk 4 with relative humidity of 95%, blotted for 3 s with a force setting of 4, and plunged into a 40/60 mixture of liquid ethane/popane⁴³ that was cooled by a bath of liquid nitrogen. Grids were transferred onto a Gatan 910 three-grid cryoholder (Gatan, Inc., CA, USA) and into the TF20 microscope maintaining a temperature no higher than −175° C. throughout. The microscope was operated at 200 kV and contrast was enhanced with a 100 μm objective aperture. Cryo-electron micrographs were collected at a nominal 62,000× magnification on the TVIPS XF416 CMOS camera with a post-column magnification of 1.3× corresponding to a calibrated pixel size of 1.8 Angstroms at the sample. Low dose methods were used to avoid electron beam damage and images were acquired with TVIPS Emplified software using movie mode for drift correction. Exposures included 10 frames at 0.15 s each for a total exposure of 1.5 s, and a total dose of approximately 10 electrons per square Angstrom.

In vitr) drug release: The release of FuOXP from FuOXP-loaded PMBOP-CP NPs with or without siRNA complexation was examined using a dialysis method. Briefly, 200 μL of FuOXP-loaded PMBOP-CP and FuOXP/siXkr8-coloaded PMBOP-CP NPs containing 200 μg of FuOXP and 2 mg of PMBOP were placed in a dialysis bag (MWCO 3.5 kDa) containing 5 mL 0.1 M PBS solution or mouse serum, respectively, and immersed into 40 mL of 0.1 M PBS solution containing 0.5% (w/v) Tween 80. The experiment was performed in an incubation shaker at 37° C. at 100 RPM. At selected time intervals, 10 μL solution in the dialysis bag and 1 mL medium outside the dialysis bag were withdrawn while same amount of fresh dialysis solution was added for replenishment. The concentration of FuOXP was examined by HPLC.

Whole-body near-infrared (NIR) fluorescence imaging and ex vivo imaging: Groups of 3 BALB/c mice were each inoculated with 5×0W CT26 cells s.c. at the right lower abdomen. When the tumors grew to ˜300 mm³, the mice were i.v. administered with Cy5.5-siXkr8-loaded PMBOP-CP NPs at a siRNA concentration of 1 mg/kg. At 12 h, 24 h and 48 h time points, the mice were imaged by IVIS 200 system (Perkin Elmer, MA, USA) at a constant is exposure time with excitation at 679 nm and emission at 702 nm for all the groups. After whole-body imaging, mice were euthanized, tumor and various organs were excised for ea vivo imaging following published protocol. For the study of PK in blood, blood was collected in Li-Heparin-containing tubes at 5 min, 0.5 h, 1 h, 2 h, 4 h, 8 h, 12 h, 48 h and 72 h time points and plasma samples were prepared by centrifugation at 12,300 RPM for 10 min and imaged by IVIS 200 system. Similar studies were performed in several other s.c. tumor models including human colon cancer (WiDr), human breast cancer (BT-474), murine pancreatic cancer (Panc02), and murine breast cancer (4T1.2, inoculated into the mammary fat pad), and an orthotopic murine colon cancer model (MC38). For evaluation of targeting efficiency in a lung metastasis model, BALB/c mice were injected with CT26-luc cells (2×10 in 100 μL. DPBS) through the tail vein. Fifteen days after tumor cell injection, whole-body and ex vivo imaging was conducted as described above. Tumor-free mice injected with Cy5.5-siRNA-loaded NPs were used as control. In addition, imaging study was conducted in CD44^{-/-} mice bearing MC38 tumors (s.c.) and compared to that in WT mice.

Blood pharmacokinetics of siXkr8: Groups of 3 naïve mice received tail vein injection of PMBOP-CP NPs loaded with FuOXP/Cy5.5-mXkr8 siRNA or free FuOXP/Cy5.5-mXkr8 siRNA at a dose of 5 and 1 mg/kg for FuOXP and siRNA, respectively. For the free drug combination, FuOXP was dissolved in Cremophor EL and mixed with siRNA prior to injection. At 5 min, 30 min, 1 h, 4 h and 24 h post injection, blood was collected and plasma was prepared. The amount of siRNA in the samples was quantified by both qRT-PCR and fluorescence measurement as detailed below.

RT-PCR: SiRNA was extracted from samples at different time points using miRNA Isolation Kit (Invitrogen, CA, USA). SuperScript III reverse transcription kit (InVitrogen, MA, USA) was used to convert siRNA into cDNA. For reverse transcription, 6 μL of RT mastermix (2 μL of water, 2 μL 5× buffer, 0.5 μL of 0.1 M DTT, 0.5 μL of 10 mM dNTPs (InVitrogen, MA, USA), 0.5 μL of RNAse OUT (InVitrogen, MA, USA), and 0.5 μL of SuperScript III enzyme) were combined with 2 μL of 0.5 μM GS primer and 2 μL of template in a 96-well plate. GS primer, and template were premixed, heated at 85° C. for 2 min, snap-chilled on ice, and RT premix was added. The 10 μL RT reaction was incubated at 50° C. for 30 min, 85° C. for 5 min, cooled to room temperature, and diluted 10-fold with 90 μL of water. Following reverse transcription, quadruplicate measurements of 2 μL of cDNA were made in 10 μL final reaction volumes by qPCR in a 384-well optical PCR plate using a 7900 HT PCR instrument (Applied Biosystems, MA, USA). SYBR green PCR mix contained 5 μL of 2× SYBR green PCR master mix (Applied Biosystems, MA, USA), 1.4 μL of water, 0.8 μL of 10 μM universal primer, 0.8 μL of 10 μM LNA-R primer, and 2 μL of sample. The primer sequences are shown in Table. 1. A standard curve was generated by spiking 100 μL of plasma aliquots from untreated animals with the NPs containing Cy5.5-mXkr8 siRNA at concentrations ranging from 0 to 20 μg/mL.

Fluorescence measurement: For this assay, an aliquot of the plasma (100 μL) was diluted with PBS to make the final volume 500 μL. Then, methanol (1.0 mL) and chloroform (0.5 mL) were added, and the samples were vortexed for 2 min until a clear, single-phase solution was obtained. The mixture was then centrifuged at 1,200 RPM for 30 min, and the aqueous phase containing Cy5.5-mXkr8 siRNA was collected. The fluorescence was measured using a Spectramax M5 multiplate reader (Molecular Devices, CA) at an excitation wavelength of679 nm and an emission wavelength of 702 nm. A standard curve was similarly generated as described above.

Biodistribution of siXkr8: Groups of3 BALB/c mice bearing s.c. CT26 tumors (˜300 mm³) received tail vein injection of PMBOP-CP NPs loaded with FuOXP/Cy5.5-mXkr8 siRNA or free FuOXP/Cy5.5-mXkr8 siRNA at a dose of5 and 1 mg/kg for FuOXP and siRNA, respectively. Hearts, livers, spleens, lungs, kidneys, and tumors were collected at 1 h, 4 h and 24 h and homogenized in 1,000 μL TRIzol™. Two-hundreds μL of chloroform was added to the homogenized tissues. After 5 min of incubation, the samples were centrifuged at 12,300 RPM for 15 min. The upper aqueous phase was similarly used for qRT-PCR and florescence quantification as described above. A standard curve was generated by spiking known amount of Cy5.5-mXkr8 siRNA (0-20 μg/mL) in the tissues obtained from non-treated control animals and used to calculate the amount of siRNA in the samples.

Quantification of platinum (Pt) by ICP-MS: Plasma and tissue samples were collected as described above. Samples were placed into a pre-weighed Purillex PFA bottle (Savillex, MN, USA) and the net weights were recorded. Plasma and tissue samples were frozen at −80° C. and lyophilized. Four (4) mL HNO₃ (69.0% w/w) and 2 mL HCl (37% w/w) were added into each PFA bottle, which was then immersed into 90° C. water bath for sample digestion to obtain free Pt ion in the lysates. The lysates were then dried down for 12 h at 50° C. to get rid of residual acid. HCl (5%) was added and the samples were transferred into a 15 mL centrifuge tube and subsequently Pt concentrations in the samples were measured using a PerkinElmer Nexion 300× Inductively Coupled Plasma-Mass Spectrometer (ICP-MS). A standard curve for Pt on the ICP-MS was created by diluting a Pt single element standard (1,000 μg/mL, for AA and ICP, Spex CertiPrep, NJ, USA).

Microscopic study of tumor distribution of NPs: For in vivo tumor biodistribution study, CT26 tumor bearing mice (˜300 mm³) were i.v. injected with Cy5.5-siRNA-loaded NPs. The mice were sacrificed at 24 h post injection. Tumor frozen sections were prepared and fixed with acetone at 4° C. for 5 min. Cytoskeleton was stained with AF488-Phalloidin (0.33 μM) (Cell Signaling Technology, MA, USA) at room temperature for 15 min and cell nuclei were stained with Hoechst 33324 (1 μg/mL) (ThermoFisher Scientific, MA, USA) at room temperature for 15 min. Tissue sections were then washed with cold DPBS three times before observation under a confocal laser scanning microscope (CLSM, FluoView 1000, Olympus, Japan).

Zombie fixation and nanoparticle circulation: NIR imaging in a zombie mouse model was conducted according to a previously published protocol. Sun, J. et al. A prodrug micellar carrier assembled from polymers with pendant farnesyl thiosalicylic acid moieties for improved delivery of paclitaxel. Acta Biomaterjalia 43, 282-291 (2016). Mice were fixed using trans-cardiac perfusion with a 4% formaldehyde and 0.5% glutaraldehyde PBS solution for 20 min. Perfusion was then performed with Cy5.5-siXkr8-loaded PMBOP-CP NPs at a siRNA concentration of22.22 μg/mL at a physiologically relevant flow rate (6 mL min⁻¹) for 6 h using a peristaltic pump. The mice were then imaged by IVIS 200 system for Cy5.5 detection.

Cellular uptake: Mouse liver sinusoidal endothelial cells (LSECs) were isolated according to a previously published protocol⁴⁷ from both WT C57BL/6 and B6.129(Cg)-Cd44^{tm1Hbg}/J (CD44^{-/-}) mice. Briefly, perfused mouse liver was cut out from the mice and grinded to release the cells. Cell suspension was then centrifuged several times at different speed and the suspended pellet was loaded on top of Percoll gradient. Non-parenchymal cells (NPC) were collected from the interface between the two density cushions of 25% with 50% Percoll and Kupffer cells were removed by selective adherence. LSECs were harvested by seeding the cells on collagen-coated cell-culture plastic dish. T cells were isolated from naïve mouse spleen and activated T cells were obtained by adding IL-2 (50 IU/mL) every two days for 1 week. For cellular uptake study, LSECs from both WT and CD44^{-/-} mice, mouse T cells (quiescent and activated) as well as sub-confluent HUVECs with or without treatment of 1% endothelial cell growth supplement (containing growth factors, hormones, and proteins for the culture of human microvascular endothelial cells) (ECGS, ScienCell, CA, USA) were incubated with Cy5.5-siRNA-loaded PMBOP-CP NPs coated with various amounts of CS/PEG-CS. Cellular uptake was examined by flow cytometry after 4 h. The expression of CD44 in LSECs, mouse T cells, and HUVECs with or without treatment of growth factors was examined by flow cytometry using both murine and human CD44-specific antibody.

In vitro gene knockdown: CT26-Luc cells, a CT26 subline stably expressing luciferase were seeded in 24-well plates in antibiotic-free DMEM/FBS. After 24 h, cells were washed with DPBS and incubated for 1 h in DMEM containing various endocytosis pathway inhibitors (Table 2), respectively. Cells were then treated with luciferase-siRNA (siLuc)-loaded PMBOP-CP NPs at a dose of 100 nM siRNA. Cells were washed at 4 h post-transfection with DPBS to remove any extracellular siRNAs and replaced with DMEM/FBS. At 24 h post-transfection, cells were collected and subjected to luciferase assay.

In vivo gene knockdown: MC38-Luc cells were s.c. inoculated into the right lower abdomen of C57BL/6 mice. SiLuc or siCT-loaded PMBOP-CP NPs were injected into MC38-Luc tumor-bearing mice at a dose of 2 mg siRNA/kg. The efficiency of gene knockdown was measured three times by whole-body bioluminescence imaging on the next day following the 1^{st}, 2^{nd} and 3^{rd} injection on day 10, 15 and 20 post tumor inoculation, respectively. Mice were anesthetized for the first two imaging and euthanized for the final imaging.

Effect of Xkr8 knockdown on the PS expression levels of tumor cells and EVs following FuOXP treatment: CT26 cancer cells were first treated with siXkr8 NPs for 72 h and then treated with FuOXP/siXkr8-coloaded NPs for another 24 h. EVs were isolated from culture medium using gradient ultra-centrifugation according to a previously established protocol⁴⁸. Briefly, culture medium was collected and centrifuged for 20 min at 1,800 RPM at 4° C. to clear dead cells and cell debris. The supernatant was then transferred to a polycarbonate ultracentrifuge tube and centrifuged at 9,000 RPM for 35 min at 4° C. to collect larger EVs. The supernatant was centrifuged again at 30,000 RPM for 80 min at 4° C. and the pellet was resuspended in 100 μL of PBS for smaller EVs collection. The levels of surface PS on tumor cells and EVs were analyzed by flow using BV421-labeled Annexin V. For detection of EVs by flow cytometry, electronic “Height” (-H) parameter rather than the “Area” (-A) parameter was used to allow optimal signal detection.

Tumor cells/macrophages co-culture study: Mouse macrophages were isolated from peritoneal cavity according to previously established protocol. CT26 cancer cells were first treated with siXkr8 NPs for 72 h and then treated with FuOXP/siXkr8-coloaded NPs for another 24 h in a 24-well plate. The treated cancer cells were then transferred and co-cultured with macrophages at a 10:1 ratio for 24 h and the macrophages were subjected to flow analysis of M1/M2 markers including F4/80 (macrophage marker) and CD206 (M2-like macrophage marker). Controls include macrophages without cancer cell co-culture, macrophages co-cultured with cancer cells without FuOXP treatment, and macrophages co-cultured with cancer cells treated with FuOXP NPs with or without coloaded siCT.

CT26 subline with stable mXkr8 knockdown: A set of 3 SMARTvector Mouse Xkr8 Lentiviral mCMV-TurboGFP shRNAs and a SMARTvector control Lentiviral mCMV-TurboGFP shRNA were purchased from Horizon Discovery Biosciences (Cambridge, UK). CT26 were transduced with each lentiviral particle and polybrene at an optimal condition and predetermined 10 MOI in 6-well plates. Cells were incubated for 24 h before sorting with TurboGFP to harvest CT26^{SMART mXkr8-/-} and CT26^{SMART}. CT26^{SMART mXkr8-/-}, CT26^{SMART} and CT26^{WT} were s.c. inoculated into the right lower abdomen of BALB/c mice and the sizes of tumors were monitored every 2 days. Tumor volume was calculated by the following formula: tumor volume=0.5×length×width².

In vivo therapeutic efficacy of FuOXP/siXkr8-coloaded NPs: CT26 or Panc02 tumor cells were s.c. inoculated into the right lower abdomen of BALB/c or C57/BL6 mice. When the tumor volume reached ˜50 mm³, mice were randomly grouped (n=5) and intravenously administered with DPBS (CT), siCT NPs, siXkr8 NPs, FuOXP NPs, FuOXP/siCT NPs or FuOXP/siXkr8 NPs three times at an interval of 5 days (day 5, 10 and 15). The doses for FuOXP and siRNA were 5 and 1 mg/kg, respectively. Mice were followed once every 2 days for tumor sizes and body weights. To test the therapeutic effect of combinational therapy of FuOXP/siXkr8 NPs with anti-PD-1, the treatment was started when the tumors reached ˜155 mm³ in sizes. Mice were treated with anti-PD-1 (clone RMP1-14, Bio X Cell, NH, USA) alone, FuOXP/siXkr8 NPs alone or the combination once every 5 days for 3 times. Anti-PD-1 was administered at 200 μg per dose intraperitoneally (i.p.) while FuOXP/siXkr8 NPs were given i.v. at a dose of 5 mg/kg for FuOXP and 1 mg/kg for siXkr8. Mice were followed until death or sacrificed if tumor size reached ˜2 cm.

Analysis of tumor-infiltrating lymphocytes and monocytes: Flow cytometry was performed with the instrument LSRII (BD Biosciences, NJ, USA) and Aurora (Cytek Biosciences, CA, USA) and analyzed by Flowlo (BD Biosciences, NJ, USA). Spleens and tumors were harvested one day after the last treatment (Day 16 post initial tumor inoculation). Single-cell suspensions were prepared from mouse spleens or tumors as previously described⁵¹. Briefly, tumors were dissected and transferred into RPMI 1640. Tumors were disrupted mechanically using scissors, digested with a mixture of deoxyribonuclease 1 (0.3 mg/mL, Sigma-Aldrich, MO, USA) and TL Liberase (0.25 mg/mL, Roche, Basal, Switzerland) in serum-free RPMI 1640 at 37° C. for 30 min, and dispersed through a 40-μm cell strainer (BD Biosciences, NJ, USA). After red blood cell lysis, live/dead cell discrimination was performed using Zombie NIR Fixable Viability Kit (BioLegend, CA, USA) at 4° C. for 30 min in DPBS. Surface staining was performed at 4° C. for 30 min in FACS staining buffer (1×phosphate-buffered saline/5% FBS/0.5% sodium azide) containing designated antibody cocktails (Annexin V. CD45, CD4, CD8, CD11b, Gr-1, F4/80, and MHC II). For intracellular proteins staining (Foxp3 and CD206), cells were fixed and permeabilized using the BD Cytofix/Cytoperm kit, following the manufacturer's instructions. For intracellular cytokine staining (IFN-γ and GzmB), cells were stimulated with phorbol 12-myristate 13-acetate (100 ng/mL) and ionomycin (500 ng/mL) for 6 h in the presence of Monensin. Cells were fixed/permeabilized using the BD Cytofix/Cytoperm kit before cell staining.

Toxicity: Body weights of mice were followed once every 2 days throughout the in vivo therapy study. After completing the experiment, blood samples were collected and ALT and AST were measured by ALT/SGPT or AST/SGOT liqui-UV assay kit following manufacturer's protocols. Tumors and major organs including heart, liver, spleen, lung, and kidney were excised and fixed in PBS containing 10% formaldehyde, followed by embedment in paraffin. The paraffin embedded samples were sectioned into slices at 4 μm using an HM 325 Rotary Microtome. The tissue slices were then subjected to H&E staining for histopathological examination under a Zeiss Axiostar plus Microscope (PA, USA).

In a separate experiment, naïve mice (n=3) received tail vein injection of siRNA-loaded PMBOP-CP NPs or siRNA complexed with DOTAP liposomes (N/P: 10/1) at a siRNA dose of 1 mg/1 kg. Two h later, blood was collected from the eye socket and the serum cytokine levels (TNF-α and IL-6) were determined with mouse cytokine assay kits.

Statistical analysis. All values were presented as mean±standard error of mean (SEM). Statistical analysis was performed with two-tailed Student's t-test for comparison between two groups and one-way analysis of variance (ANOVA) for comparison between multiple groups. Results were considered statistically significant if p<0.05.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A therapeutic system or combination comprising a first therapeutic agent to treat a disease condition and a second therapeutic agent administrable within a predetermined time of administration of the first therapeutic agent, the second therapeutic agent inhibiting the function of Xkr8.

2. The therapeutic system of claim 1 wherein the first therapeutic agent induces a therapeutic response which induces the expression of Xkr8 in addition to activation of Xkr8.

3. The therapeutic system of claim 1 wherein the second therapeutic agent functions via RNA interference.

4. The therapeutic system of claim 1 wherein the second therapeutic agent comprises Xkr8 siRNA.

5. The therapeutic system of claim 4 wherein the first therapeutic agent is a chemotherapeutic agent.

6. The therapeutic system of claim 4 wherein the system comprises nanostructures formed from self-assembly of a plurality of amphiphilic polymers comprising cationic groups, wherein a plurality of the first therapeutic agent, which is a hydrophobic or a lipophilic chemotherapeutic agent, is associated with a core of the nanostructures, and the second therapeutic agent comprising Xkr8 siRNA is added to the nanostructures and is associated with the cationic groups thereof.

7. The therapeutic system of claim 6 wherein the nanostructures further comprise an application added to the nanostructures, the application comprising a negatively charged targeting agent selected from the group of a ligand for a cell receptor, a peptide, an aptamer, a polysaccharide, and an antibody.

8.-13. (canceled)

14. The therapeutic system of claim 2 wherein the targeting agent is a cell receptor and the cell receptor is a CD44 ligand.

15. The therapeutic system of claim 14 wherein the application further comprises a hydrophilic polymeric compound.

16. The therapeutic system of claim 15 wherein the hydrophilic polymeric compound includes a negative charge.

17. The therapeutic system of claim 16 wherein the hydrophilic polymeric compound includes a conjugate of a negatively charged molecule and a hydrophilic polymer.

18. The therapeutic system of claim 17 wherein the negatively charged molecule conjugated to the hydrophilic polymer is a CD44 ligand.

19. The therapeutic system of claim 18 wherein the CD44 ligand is osteopontin, a collagen, a matrix metalloproteinase, chondroitin sulfate, hyaluronic acid, or a derivative thereof.

20. (canceled)

21. The therapeutic system of claim 15 wherein the second therapeutic agent comprising Xkr8 siRNA is added to the nanostructures before application of the negatively charged CD44 ligand and the hydrophilic polymeric compound.

22. The therapeutic system of claim 15 wherein the first therapeutic agent is a small molecule therapeutic compound having a molecular weight below 1 kDa.

23. (canceled)

24. The therapeutic system of claim 15 wherein the cationic groups comprise an inherently cationic group or a group which forms a cation in vivo.

25. The therapeutic system of claim 24 wherein the group which forms a cation in vivo is an amine group, wherein the amine group is an acyclic amine group, a cyclic amine group or a heterocyclic amine group.

26. The therapeutic system of claim 25 wherein the amine group is selected from the group consisting of a metformin group, a morpholine group, a piperazine group, a pyridine group, a pyrrolidine group, piperidine, a thiomorpholine, a thiomorpholine oxide, a thiomorpholine dioxide, an imidazole, a guanidine, a biguanidine or a creatine.

27. The therapeutic system of claim 17 wherein the hydrophilic polymer is selected from the group consisting of a polyalkylene oxide, a polyvinylalcohol, a polyacrylic acid, a polyacrylamide, a polyoxazoline, a polysaccharide and a polypeptide.

28. The therapeutic system of claim 17 wherein the hydrophilic polymer is polyethylene glycol.

29. The therapeutic system of claim 15 wherein a ratio of the negatively charged CD44 ligand to the hydrophilic polymeric compound is determined such that uptake of the nanostructures in the liver of a patient is maintained at a sufficiently low level to allow interaction of the negatively charged CD44 ligand with CD44 on a tumor remote from the liver.

30. The therapeutic system of claim 16 wherein each of the plurality of amphiphilic polymers comprises a hydrophobic polymer backbone, a first plurality of pendant groups attached to the hydrophobic polymer backbone and comprising at least one of the cationic groups, and a second plurality of pendant groups attached to the hydrophobic polymer backbone and comprising at least one hydrophilic polymer.

31. The therapeutic system 30 wherein the hydrophobic polymer backbone further comprises a pendant lipidic group.

32.-52. (canceled)

53. A formulation, comprising: nanostructures formed from self-assembly of a plurality of amphiphilic polymers, a plurality of a first hydrophobic or lipophilic therapeutic agent associated with a core of each of the nanostructures, and a second therapeutic agent which decreases the Xkr8 expression level or inhibits the function of Xkr8.

54.-88. (canceled)
